(19) United States
(12) Patent Application Publication
(10) Pub. No.: US 2021/0100826 A1
(43) Pub. Date: Apr. 8, 2021

(54) ACTIVATION OF P2X7 RECEPTORS WITH NON-BZBZ ADENOSINE TRIPHOSPHATE DERIVATIVES

(71) Applicant: UNIVERSITY HOSPITAL CLEVELAND MEDICAL CENTER, Cleveland, OH (US)

(72) Inventor: Nathan Bryson, Toronto (CA)

(21) Appl. No.: 16/500,325

(22) PCT Filed: Apr. 3, 2018
(86) PCT No.: PCT/US18/25954

§ 371 Date: Oct. 2, 2019

Related U.S. Application Data
(60) Provisional application No. 62/480,714, filed on Apr. 3, 2017.

(51) Int. Cl. A61K 31/7076

(52) U.S. Cl. CPC A61K 31/7076

(57) ABSTRACT

Compositions and methods are presently disclosed that provide improved efficiacy for the treatment of cancer. In particular, 3-O-ribose monoester derivatives of adenosine triphosphate are observed to have vastly superior efficiacy over a previously reported compound, 3,5 benzoylbenzoyl adenosine triphosphate. These novel compounds have been shown to increase calcium channel activation mediated by the P2X7 receptor thereby resulting in increase apoptosis of cancer cells, either malignant or benign.

### FIELD OF THE INVENTION

The present invention is related to the field of treatment and prevention of cancer. For example, the compositions and methods herein are related to intracellular apoptosis induction by P2X7 receptor activation. Some compositions that induce P2X7 receptor activation comprise adenosine triphosphate (ATP) derivatives. Some methods that treat and/or prevent cancer comprise the administration of ATP derivatives.

### BACKGROUND OF THE INVENTION

Cancer is a disease having many etiologies encompassing environmental toxins, disease, microbiological infections, and/or genetic predispositions. As such, each causative factor can, and does, result in a different type of cancer that usually manifests in a different biological tissue. As a result, no one therapeutic approach has been identified that has been effective at slowing or preventing the progression of a large percentage of different cancerous types. The only commonality that is currently recognized between all cancer diseases is manifested by an uncontrolled cellular growth rate.

Current theories related to epithelial cell growth predicts a regulatory pathway that balances the effects of mitogenic stimuli and apoptosis. Croker et al., “Cancer stem cells: implications for the progression and treatment of metastatic disease” J Cell Mol Med 2008, 12:374-390; and Rodriguez-Nieto et al., “Role of alterations in the apoptotic machinery in sensitivity of cancer cells to treatment’ Curr Pharm Des 2006, 12:4411-4425. Apoptosis is believed to be a homeostatic process orchestrated by the host's genome of selective cell deletion without stimulating inflammatory response. Wyllie et al., “Cell death: the significance of apoptosis” Int Rev Cytol 1980, 68:251-306; Ellis et al., “Mechanisms and functions of cell death” Annul Rev Cell Biol 1991, 7:663-698; and Fawthrop et al., “Mechanisms of cell death” Arch Toxicol 1991, 65:437-444. Dysregulation of apoptotic cell-death has been implicated in states of disease and in the neoplastic transformation. Soti et al., “Apoptosis, necrosis and cellular senescence: chaperone occupancy as a potential switch” Aging Cell 2003, 2:39-45; and Renvoize et al., “Apoptosis: identification of dying cells” Cell Biol Toxicol 1998, 14:111-120. Present anti-cancer therapies all share a common problem in that nonnal non-cancerous cells are susceptible to the various treatments (i.e., for example, radiation and/or chemotherapy).

What is needed in the art are improved compositions to treat cancer. One approach is to provide more efficacious adenosine triphosphate compounds that enhance the induction of apoptosis by P2X7 receptor activation.

### SUMMARY OF THE INVENTION

The present invention is related to the field of treatment and prevention of cancer. For example, the compositions and methods herein are related to intracellular apoptosis induction by P2X7 receptor activation. Some compositions that induce P2X7 receptor activation comprise adenosine triphosphate (ATP) derivatives. Some methods that treat and/or prevent cancer comprise the administration of ATP derivatives.

In one embodiment, the present invention contemplates a method, comprising: a) providing: i) a subject comprising at least one cancer cell; and ii) a composition comprising a non-benzoylbenzoyl adenosine triphosphate derivative (ATPd); and b) administering said composition to said subject wherein said at least one cancer cell undergoes apoptosis. In one embodiment, the at least one cancer cell is a malignant cancer cell. In one embodiment, the at least one cancer cell is a benign cancer cell. In one embodiment, the at least one cancer cell comprises a papilloma cancer cell. In one embodiment, the at least one cancer cell comprises an epithelial cancer cell. In one embodiment, the ATPd is a 3-O-ribose monoester ATPd. In one embodiment, the ATPd is benzoyl-ATP. In one embodiment, the ATPd is lauroyl-ATP. In one embodiment, the ATPd is phenoxybenzoyl-ATP. In one embodiment, the ATPd is cinnamoyl-ATP. In one embodiment, the administering includes, but is not limited to local administration such as topical, intradermal, intratumoral, intranasal and/or transdermal. In one embodiment, the administering includes, but is not limited to parenteral administration such as intraperitoneal, intravenous, intramuscular, and/or subcutaneous. In one embodiment, the administering is oral.

In one embodiment, the present invention contemplates a method, comprising: a) providing: i) a subject comprising at least one tumor; and ii) a composition comprising a non-benzoylbenzoyl adenosine triphosphate derivative (ATPd); and b) administering said composition to said subject wherein said at least one tumor undergoes a regression. In one embodiment, the regression is partial. In one embodiment, the partial regression is between approximately 10% to 90%. In one embodiment the regression is complete. In one embodiment, the complete regression is 100%. In one embodiment, the at least tumor is a malignant tumor. In one embodiment, the at least tumor is a benign tumor. In one embodiment, the at least one tumor comprises a papilloma. In one embodiment, the at least tumor comprises an epithelial tumor. In one embodiment, the ATPd is a 3-O-ribose monoester ATPd. In one embodiment, the ATPd is benzoyl-ATP. In one embodiment, the ATPd is lauroyl-ATP. In one embodiment, the ATPd is phenoxybenzoyl-ATP. In one embodiment, the ATPd is cinnamoyl-ATP. In one embodiment, the administering includes, but is not limited to local administration such as topical, intradermal, intratumoral, intranasal and/or transdermal. In one embodiment, the administering includes, but is not limited to parenteral administration such as intraperitoneal, intravenous, intramuscular, and/or subcutaneous. In one embodiment, the administering is oral.

In one embodiment, the present invention contemplate a pharmaceutical composition comprising a non-benzoylbenzoyl adenosine triphosphate derivative (ATPd) and a pharmaceutically acceptable carrier. In one embodiment, the ATPd is a 3-O-ribose monoester ATPd. In one embodiment, the ATPd is benzoyl-ATP. In one embodiment, the ATPd is lauroyl-ATP. In one embodiment, the ATPd is phenoxybenzoyl-ATP. In one embodiment, the ATPd is cinnamoyl-ATP. In one embodiment, the pharmaceutically acceptable carrier is a semi-solid medium. In one embodiment, the pharmaceutically acceptable carrier is a liquid medium. In one embodiment, the pharmaceutically acceptable carrier comprises a liposome. In one embodiment, the pharmaceutically acceptable carrier comprises a microparticle. In one embodiment, the ATPd is encapsulated by the liposome. In one embodiment, the ATPd is attached to the microparticle.

In one embodiment, the present invention contemplate a kit comprising: a) a first container comprising a pharmaceutical composition comprising a non-benzoylbenzoyl adenosine triphosphate derivative (ATPd) and a pharmaceutically acceptable carrier; and b) a set of instructions regarding a method to treat cancer cells with the pharmaceutical composition. In one embodiment, the ATPd is a 3-O-ribose monoester ATPd. In one embodiment, the ATPd is benzoyl-ATP. In one embodiment, the ATPd is lauroyl-ATP. In one embodiment, the ATPd is phenoxybenzoyl-ATP. In one embodiment, the ATPd is cinnamoyl-ATP. In one embodiment, the pharmaceutically acceptable carrier is a semi-solid medium. In one embodiment, the pharmaceutically acceptable carrier is a liquid medium. In one embodiment, the pharmaceutically acceptable carrier comprises a liposome. In one embodiment, the pharmaceutically acceptable carrier comprises a microparticle. In one embodiment, the ATPd is encapsulated by the liposome. In one embodiment, the ATPd is attached to the microparticle.

### Definitions

The term “cancer”, as used herein refers to any presence of cells possessing characteristics typical of cancer-causing cells, for example, uncontrolled proliferation, loss of specialized functions, immortality, significant metastatic potential, significant increase in anti-apoptotic activity, rapid growth and proliferation rate, and certain characteristic morphology and cellular markers. In some circumstances, cancer cells will be in the form of a tumor; such cells may exist locally within an animal, or circulate in the blood stream as independent cells, for example, leukemia cells. The number of cancer cells in a subject's body can be determined by direct measurement, or by estimation from the size of primary or metastatic tumor masses. For example, the number of cancer cells in a subject can be measured by immunohistological methods, flow cytometry, or other techniques designed to detect characteristic surface markers of cancer cells.

The term “salts”, as used herein, refers to any salt or counterion that complexes with identified compounds contained herein while retaining a desired function, e.g., biological activity.

The term “counterion”, as used herein, refers to the ion that accompanies an ionic species in order to maintain electric neutrality. Examples of such salts include, but are not limited to, acid addition salts formed with inorganic acids (e.g. hydrochloric acid, hydrobromic acid, sulfuric acid, phosphoric acid, nitric acid, and the like), and salts formed with organic acids such as, but not limited to, acetic acid, oxalic acid, tartaric acid, succinic acid, malic acid, fumaric acid, maleic acid, ascorbic acid, benzoic acid, tannic acid, pamoic acid, alginic acid, polyglutamic, acid, naphthalene sulfonic acid, naphthalene disulfonic acid, and polygalacturonic acid, Pharmaceutically acceptable salts also include base addition salts which may be formed when acidic protons present are capable of reacting with inorganic or organic bases. Suitable pharmaceutically-acceptable base addition salts include metallic salts, such as salts made from aluminum, calcium, lithium, magnesium, potassium, sodium and zinc, or salts made from organic bases including primary, secondary and tertiary amines, substituted amines including cyclic amines, such as caffeine, arginine, diethylamine, N-ethyl piperidine, histidine, glucamine, isopropylamine, lysine, morpholine, N-ethyl morpholine, piperazine, piperidine, triethylamine, trimethylamine. All of these salts may be prepared by conventional means from the corresponding compound of the invention by reacting, for example, the appropriate acid or base with the compound of the invention. Unless otherwise specifically stated, the present invention contemplates pharmaceutically acceptable salts of the considered compositions.

The term “tumor” or “papilloma” as used herein, refers to all neoplastic cell growth and proliferation, whether malignant or benign. The size of a tumor can be ascertained by direct visual observation, or by diagnostic imaging methods, including, but not limited to, X-ray, magnetic resonance imaging, ultrasound, and scintigraphy. Diagnostic imaging methods used to ascertain size of the tumor can be employed with or without contrast agents. The size of a tumor can also be ascertained by physical means, such as palpation of the tissue mass or measurement of the tissue mass with a measuring instrument, such as a caliper.

The term “cancer symptoms” as used herein, refers to observable changes in a subject's physical and/or medical condition consistent with a specific type of cancer. In general, cancer symptoms may include, but are not limited to, weight loss, fatigue, localized swelling, or localized pain. Each cancer type comprises symptoms that may or may not occur in a different type of cancer. For example, symptoms of uterine cancer include, but are not limited to, abnormal bleeding, spotting, or other discharges from the vagina. On the other hand, symptoms of cervical cancer include, but are not limited to, continuous vaginal discharge, abnormal and/or heavy vaginal bleeding, loss of appetite, pelvic and/or back pain, single swollen leg, or bone fractures.

The term “local” as used herein, refers to one route of a non-parenteral administration of a therapeutic agent. A local administration may include, but is not limited to topical or intratumoral. A minimal amount of systemic distribution is expected during a local administration but would be expected to maintain subclinical thresholds.

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “regression” as used herein, in regards to a tumor means any progressive decline of any manifestation of the tumor. For example, a tumor manifestation may include, but not limited to, a reduction in size, volume, height, diameter, density and/or severity. When a tumor regession is expressed in percent, it is understood that the percent regression is relative to the pre-treatment tumor manifestations as compared to the post-treatment manisfestations.

The term “attached” as used herein, refers to any interaction between a medium (or carrier) and a drug. Attachment may be reversible or irreversible. Such attachment includes, but is not limited to, covalent bonding, ionic bonding, Van der Waals forces or friction, and the like. A drug is attached to a medium (or carrier) if it is impregnated, incorporated, coated, in suspension with, in solution with, mixed with, etc.

The term “medium” as used herein, refers to any material, or combination of materials, which serve as a carrier or vehicle for delivering of a drug to a treatment point (e.g., wound, surgical site etc.). For all practical purposes, therefore, the term “medium” is considered synonymous with the term “carrier”. It should be recognized by those having skill in the art that a medium comprises a carrier, wherein said carrier is attached to a drug or drug and said medium facilitates delivery of said carrier to a treatment point. Further, a carrier may comprise an attached drug wherein said carrier facilitates delivery of said drug to a treatment point. Preferably, a medium is selected from the group including, but not limited to, foams, gels (including, but not limited to, hydrogels), xerogels, microparticles (i.e., microspheres, liposomes, microcapsules etc.), bioadhesives, or liquids. Specifically contemplated by the present invention is a medium comprising combinations of microparticles with hydrogels, bioadhesives, foams or liquids. Preferably, hydrogels, bioadhesives and foams comprise any one, or a combination of, polymers contemplated herein. Any medium contemplated by this invention may comprise a controlled release formulation. For example, in some cases a medium constitutes a drug delivery system that provides a controlled and sustained release of drugs over a period of time lasting approximately from 1 day to 6 months.

The term “administered” or “administering” a drug or composition, as used herein, refers to any method of providing a drug or compound to a patient such that the drug or compound has its intended effect on the patient. For example, one method of administering is by an indirect mechanism using a medical device such as, but not limited to a catheter, applicator gun, syringe etc. A second exemplary method of administering is by a direct mechanism such as, local tissue administration (i.e., for example, extravascular placement), oral ingestion, transdermal patch, topical, inhalation, suppository etc.

The term “subject” and/or “patient”, as used herein, is a human or animal and need not be hospitalized. For example, out-patients, persons in nursing homes are “subjects” and/or “patients.” A patient may comprise any age of a human or non-human animal and therefore includes both adult and juveniles (i.e., children). It is not intended that the terms “subject” and/or “patient” connote a need for medical treatment and therefore may voluntarily or involuntarily be part of experimentation whether clinical or in support of basic science studies.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, and the like. Supplementary bioactive ingredients also can be incorporated into such carriers.

The term “biodegradable” as used herein, refers to any material that can be acted upon biochemically by living cells or organisms, or processes thereof, including water, and broken down into lower molecular weight products such that the molecular structure has been altered.

The term “bioerodible” as used herein, refers to any material that is mechanically worn away from a surface to which it is attached without generating any long term inflammatory effects such that the molecular structure has not been altered. In one sense, bioerosin represents the final stages of “biodegradation” wherein stable low molecular weight products undergo a final dissolution.

The term “bioresorbable” as used herein, refers to any material that is assimilated into or across bodily tissues. The bioresorption process may utilize both biodegradation and/or bioerosin.

The term “biostable” as used herein, refers to any material that remains within a physiological environment for an intended duration resulting in a medically beneficial effect.

The term “small organic molecule” as used herein, refers to any molecule of a size comparable to those organic molecules generally used in pharmaceuticals. The term excludes biological macromolecules (e.g., proteins, nucleic acids, etc.). Preferred small organic molecules range in size from approximately 10 Da up to about 5000 Da, more preferably up to 2000 Da, and most preferably up to about 1000 Da.

The term “a cell comprising a P2X₇ receptor” as used herein, refers to any cell derived from a bodily tissue displaying a P2X₇ receptor. wherein activation of the receptor induces apoptosis. For example, such cell include, but are not limited to, epithelial cells, neuronal cells, glial cells, endothelial cells, bone marrow cells, muscle cells, hemopoietic cells, white blood cells, gastrointestinal cells, urinary tract cells, gonadal cells, renal cells, pancreatic cells, retinal cells, prostate cells, lung cells, or kidney cells.

The term “derivative”, as used herein refers to any chemically modification of a core structure. For example, an adenosine triphosphate molecule may be chemically modified to create adenosine triphosphate derivatives (ATPd's). For example, such chemical modifications may comprise a 3-O-ribose monoester modification. Such ATPd's may include, but are not limited to, benzoyl, cinnamoyl, phenoxybenzoyl, and/or lauroyl chemical modifications. In contrast, a benzoylbenzoyl adenosine triphosphate (BzBzATP), as used herein, is not contemplated herein as an ATPd.

### BRIEF DESCRIPTION OF THE FIGURES

The file of this patent contains at least one drawing executed in color. Copies of this patent with color drawings will be provided by the Patent and Trademark Office upon request and payment of the necessary fee.

FIG. 1 presents illustrative photographs of gross morphology of skin papillomas in DMBA/TPA− and in DMBA/TPA+BzBzATP-treated mice. FIGS. 1A and 1B represent hematoxylin/eosin (H&E) staining. FIGS. 1C and 1D represent TUNEL staining (×10). Arrows in FIG. 1C point to papillomas at various stages of involution. Arrow in FIG. 1D points to increased TUNEL staining in basal/parabasal layers of outgrowing keratinocytes in the papilloma.

FIG. 2 presents representative photographs of DMBA/TPA-induced skin lesions in mice in-vivo, and the effects of co-treatment with BzBzATP. Arrows in FIG. 2D and FIG. 2 E point to involuting papillomas.

FIG. 3 presents representative histological cross-sections, evaluated histologically by H&E staining, of DMBA/TPA-induced skin lesions in mice in-vivo, and the effects of co-treatment with BzBzATP.

FIG. 4 presents exemplary data showing a summary of the effects of local treatments with DMBA/TPA (black symbols) or DMBA/TPA+BzBzATP (white symbols) on the proportion of living mice with skin lesions. (expressed as mean data; standard deviation (SD) ranges between 3-11%).

FIG. 4A: Skin lesions at 0-12 weeks of treatment were papillomas. Skin lesions at 14-28 weeks of treatment were grouped either as cancerous lesions (squamous spindle-cell carcinomas, circles), or as non-cancerous lesions (existing or involuting papillomas, triangles).

FIG. 4B: Skin lesions at 14-28 weeks of treatment were grouped either as cancerous lesions (squamous spindle-cell carcinomas, circles), or as non-cancerous lesions (existing or involuting papillomas, triangles).

FIGS. 5A-5C present exemplary data showing a summary of the effects of local treatments with DMBA/TPA (black symbols) or DMBA/TPA+BzBzATP (white symbols) on the mean number of skin lesions per living animal. Values represent means, and standard deviations ranged between 5-9%.

FIG. 6 presents exemplary data showing a summary of the effects of local treatments with DMBA/TPA (black symbols) or DMBA/TPA+BzBzATP (white symbols). Values represent means, and standard deviations ranged between ranged 2-18%.

FIG. 6A: Mean lesion size between 0-12 weeks.

FIG. 6B: Proportion of living mice with total lesions volume per animal of >10 mm³.

FIG. 6C: Proportion of living mice with total lesions volume per animal of >200 mm³.

FIG. 7 presents illustrative embodiments of the synthesis pathway for non-benzoylbenzoyl-ATP derivatives (ATPds). For example, an ATP-disodium salt is used as a starting material that leads to the formation of ATDd's including, but not limited to, lauroyl-ATP, benzoyl-ATP and/or 3-phenoxybenzoyl-ATP.

FIG. 8 presents exemplary data of an electrochemical patch clamp assay dose response curve for NaATP.

FIG. 9 presents exemplary data of an electrochemical patch clamp assay dose response curve for previously reported BzBz-ATP.

FIG. 10 presents exemplary data of an electrochemical patch clamp assay dose response curve for lauroyl-ATP solubilized in dimethylsulfoxide (DMSO).

FIG. 11 presents exemplary data of an electrochemical patch clamp assay dose response curve for 3-phenoxybenzoyl-ATP.

FIG. 12 presents exemplary data of an electrochemical patch clamp assay dose response curve for benzoyl-ATP.

### DETAILED DESCRIPTION OF THE INVENTION

The present invention is related to the field of treatment and prevention of cancer. For example, the compositions and methods herein are related to intracellular apoptosis induction by P2X7 receptor activation. Some compositions that induce P2X7 receptor activation comprise adenosine triphosphate (ATP) derivatives. Some methods that treat and/or prevent cancer comprise the administration of ATP derivatives.

Anti-apoptotic mechanisms may contribute to the development of cancer. The P2X₇ system is believed to be a pro-apoptosis modulator in epithelial cells, and augmentation of P2X₇-mediated apoptosis has been proposed as a pharmacological modality for chemoprevention and treatment of epithelial cancers.

The growing understanding of mechanisms of P2X₇-mediated apoptosis has generated a strategy for targeting directly and specifically skin neoplasia. Although it is not necessary to understand the mechanism of an invention, it is believed that the data presented herein link directly, for the first time, an up-regulation of apoptosis with cancer prevention and treatment. For example, significant antitumor efficacy has been achieved in a rodent cancer model, and it is likely that compounds affecting P2X₇-control of apoptosis are useful for preventing and treating cancer (i.e., for example, epithelial cancer).

### I. Cancer

Cancer is believed to be a disease of overproliferation, wherein the present invention provides a method to reduce this overproliferation. Cancerous cells are also called malignant cells and are derived from normal cells in the body. Cancer appears to occur when the growth of cells in the body is out of control and cells divide too quickly. It can also occur when cells “forget” how to die (i.e., for example, reduced apoptosis). There are many different kinds of cancers. Cancer can develop in almost any organ or tissue, including, but not limited to, the lung, colon, breast, skin, bones, or nerve tissue.

There are many causes of cancers, including, but not limited to, benzene and other chemicals, poisonous mushrooms and a type of poison that can grow on peanut plants (i.e., for example, aflatoxins), viruses, radiation, sunlight, or tobacco. However, the cause of many cancers remains unknown. The most common cancers in men in the United States include, but are not limited to skin cancer, prostate cancer, lung cancer, and colon cancer. In women in the U.S., the most common cancers include, but are not limited to, breast cancer, skin cancer, lung cancer, and colon cancer.

Some other types of cancers include, but are not limited to, brain cancer, cervical cancer, Hodgkin's lymphoma, kidney cancer, leukemia, liver cancer, Non-Hodgkin's lymphoma, ovarian cancer, skin cancer, testicular cancer, thyroid cancer, or uterine cancer. Symptoms of cancer depend on the type and location of the tumor. For example, lung cancer can cause coughing, shortness of breath, or chest pain. Colon cancer often causes diarrhea, constipation, and blood in the stool. Some cancers may not have any symptoms at all. In certain cancers, such as gallbladder cancer, symptoms often do not start until the disease has reached an advanced stage. The following symptoms can occur with most cancers: chills, fatigue, fever, loss of appetite, malaise, night sweats, or weight loss.

Common tests to identify cancer may include, but are not limited to, biopsy, blood chemistries x-ray, complete blood count, computerized tomography scan, or magnetic resonance imaging scan.

Conventional treatment varies based on the type of cancer and its stage. The stage of a cancer refers to how much it has grown and whether the tumor has spread from its original location. If the cancer is confined to one location and has not spread, current treatments are oriented towards surgery, radiation and/or chemotherapy. This is often the case with skin cancers, as well as cancers of the lung, breast, and colon.

Epithelial cancers are common and usually display aggressive and fatal biological-clinical behavior. Epithelia are tissues that line body surfaces. Although it is not necessary to understand the mechanism of an invention, it is believed that the present invention will lead to better understanding of how epithelial cancers develop. In one embodiment, the present invention contemplates a method for detecting cancers at early stage of development, consequently resulting in earlier treatment and improved survival rates. In one embodiment, the present invention contemplates methods of treating epithelial cancers. In one embodiment, the present invention contemplates methods of preventing epithelial cancers (i.e., for example, prophylactic treatments).

Epithelial cancers are thought to be common and can display aggressive and potentially fatal biological clinical behavior. Although it is not necessary to understand the mechanism of an invention, it is believed that some embodiments of the present invention could lead to: i) improved understanding of epithelial cancer development; ii) improved early cancer detection; iii) improved early cancer treatment; iv) new modalities and directions for cancer treatments; and v) improved epithelial cancer prevention.

Cancer development is believed associated with inactivation of tumor-controlling genes, including tumor suppressor and apoptosis-related genes. Inactivation of genes can be the result of allelic loss or loss-of-heterozygosity chromosomal sites due to gene mutations, deletions, and genomic rearrangements. Some cancers exhibit a number of genomic alterations including monoallelic hemizygous deletions at 4p15.3, 10q24, 5q35, 3p12.3, and 11q24. Wistuba et al., “Deletions of chromosome 3p are frequent and early events in the pathogenesis of uterine cervical carcinoma” Cancer Res. 57:3154-3158 (1997); Chu et al., “Monoclonality and surface lesion specific microsatellite alterations in premalignant and malignant neoplasia of uterine cervix: a local field effect of genomic instability and clonal evolution” Genes Chromosomes Cancer 24:127-134 (1999); and Hamoudi et al., “Identification of novel prognostic markers in cervical intraepithelial neoplasia using 1DMAS (loh data management and analysis software)” BMC Bioinformatics 6:18 (2005). Alternatively, other studies demonstrate a possible loss of tumor suppressor gene on chromosome 11q23. Lai et al., “Hypermethylation of two consecutive tumor suppressor genes, BLU and RASSFIA, located at 3p21.3 in cervical neoplasias” Gynecol Oncol. 104:629-635 (2007).

### II. Apoptosis

The current theory of epithelial cell growth predicts regulation by the concerted actions of mitogenic stimuli and apoptosis. Croker et al., “Cancer stem cells: implications for the progression and treatment of metastatic disease” J Cell Mol Med 2008; 12:374-90; and Rodriguez-Nieto et al., “Role of alterations in the apoptotic machinery in sensitivity of cancer cells to treatment” Curr Pharm Des 2006; 12:4411-25. Apoptosis is a homeostatic process orchestrated by the host's genome of selective cell deletion without stimulating inflammatory response. Wyllie et al., “Cell death: the significance of apoptosis” Int Rev Cytol 1980; 68:251 306; Ellis et al., “Mechanisms and functions of cell death” Annul Rev Cell Biol 1991; 7:663 98; and Fawthrop et al., “Mechanisms of cell death” Arch Toxicol 1991; 65:437-44. Earlier studies showed that apoptosis is activated in response to noxious stimuli e.g. starvation, inflammation, infection, irradiation, etc. More recent data suggested a physiological role for apoptosis, including the control of tissue development and differentiation, regulation of mitogenic effects, and control of cell death and loss of tissue with aging, and dysregulation of apoptotic cell-death has been implicated in states of disease. Soti et al., “Apoptosis, necrosis and cellular senescence: chaperone occupancy as a potential switch” Aging Cell 2003; 2:39-45.

Apoptosis is believed to be a process of programmed cell death that may occur in multicellular organisms. Programmed cell death involves a series of biochemical events leading to a characteristic cell morphology and death, in more specific terms, a series of biochemical events that lead to a variety of morphological changes, including blebbing, changes to the cell membrane such as loss of membrane asymmetry and attachment, cell shrinkage, nuclear fragmentation, chromatin condensation, and chromosomal DNA fragmentation. Processes of disposal of cellular debris whose results do not damage the organism differentiate apoptosis from necrosis.

In contrast to necrosis, which is a form of traumatic cell death that results from acute cellular injury, apoptosis, in general, confers advantages during an organism's life cycle. For example, the differentiation of fingers and toes in a developing human embryo occurs because cells between the fingers apoptose; the result is that the digits are separate. Between 50 billion and 70 billion cells die each day due to apoptosis in the average human adult. For an average child between the ages of 8 and 14, approximately 20 billion to 30 billion cells die a day. In a year, this amounts to the proliferation and subsequent destruction of a mass of cells equal to an individual's body weight. Excessive apoptosis causes hypotrophy, such as in ischemic damage, whereas an insufficient amount results in uncontrolled cell proliferation, such as cancer.

Apoptosis may occur when a cell is damaged beyond repair, infected with a virus, or undergoing stressful conditions such as starvation. Damage to DNA from ionizing radiation or toxic chemicals can also induce apoptosis via the actions of the tumour-suppressing gene p53. The “decision” for apoptosis can come from the cell itself, from the surrounding tissue, or from a cell that is part of the immune system. In these cases, apoptosis functions to remove the damaged cell, preventing it from sapping further nutrients from the organism, or halting further spread of viral infection.

As discussed further below, apoptosis may also play a role in preventing cancer. If a cell is unable to undergo apoptosis because of mutation or biochemical inhibition, it continues to divide and may develop into a tumor. For example, infection by papillomaviruses causes a viral gene to interfere with the cell's p53 protein, an important member of the apoptotic pathway. This interference in the apoptotic capability of the cell plays a role in the development of cervical cancer.

In an adult organism, the number of cells is kept relatively constant through cell death and division (i.e., proliferation). Cells must be replaced when they malfunction or become diseased, but proliferation must be offset by cell death. This control mechanism is part of the homeostasis required by living organisms to maintain their internal states within certain limits. Homeostasis is achieved when the rate of mitosis (cell division) in the tissue is balanced by cell death. If this equilibrium is disturbed, one of two potentially fatal disorders may occur: i) the cells are dividing faster than they die, effectively developing a tumor; or ii) the cells are dividing slower than they die, causing cell loss.

Homeostasis involves a complex series of reactions, an ongoing process inside an organism that calls for different types of cell signaling. Any impairment can cause a disease. For example, dysregulation of signaling pathway has been implicated in several forms of cancer. The pathway, which conveys an anti-apoptotic signal, has been found to be activated in pancreatic adenocarcinoma tissues.

A. Mechanisms of Apoptosis

Histologically, apoptosis may be characterized by DNA fragmentation, chromatin condensation, membrane blebbing, cell detachment from the extracellular matrix, cell rounding and shrinking, and alterations in plasma membrane lipid organization. Usually, the final stages of apoptosis are induced by a series of proteolytic enzymes termed caspases, which cleave and activate each other in a cascade of proteolysis, terminating with the effector caspases 7 and 3. Boatright et al., “Mechanisms of caspase activation” Curr Opin Cell Biol 2003; 15:725-31; and Klein et al., “Killing time for cancer cells” Nat Rev Cancer 2005; 5:573-580

Several cellular pathways are involved in the activation of the caspase family of proteases and the induction of apoptosis. In one embodiment, the present invention contemplates a method wherein apoptosis may involve pathways including, but not limited to: a) the intrinsic mitochondrial pathway; or b) the extrinsic death-receptor pathway. Lorenzo et al., “Therapeutic potential of AIF-mediated caspase independent programmed cell death” Drug Resist Updates 2007; 10:235-55.

Apoptosis via the intrinsic pathway is characterized predominantly by mitochondrial changes. Effects are triggered by stimuli that cause mitochondrial disturbances and DNA damage (such as cancer therapeutic agents and ionizing irradiation), oxidative stress, hypoxia, cell detachment, and cellular distress. Degterev et al., “A decade of caspases” Oncogene 2003; 22:8543-8567. Signals from these diverse stimuli converge upon the mitochondria, where propagation of the apoptotic signal is regulated by proteins that either promote (e.g. Bax, Bak, Bok, Bad, Bid, Bik, Bim, Bel-Xs, Krk, Mtd, Nip3, Nix, Noxa, and Bcl-B) or suppress apoptosis (e.g. Bcl-2, Bel-XL, Mel-1, Bfl-1/A1, Bel-W, and Bel-G). Guo et al., “Bcl-G, a novel pro-apoptotic member of the Bcl-2 family” J Biol Chem 2001; 276:2780-2785; and Antonsson et al., “The Bcl-2 protein family” Exp Cell Res 2000; 256:50-57.

Pro-apoptotic signals trigger permeabilization of the mitochondrial outer membrane, and facilitate the release of proteins from the mitochondrial intermembranous space into the cytoplasm, including cytochrome c and Smac/Diablo. The released cytochrome c then binds the caspase adaptor apoptotic protease-activating factor-1 (Apaf-1), thereby activating procaspase 9 and forming the apoptosome complex. Green D R., “Apoptotic pathways: ten minutes to dead” Cell 2005; 121:671-674. The apoptosome activates several downstream effector caspases, such as caspases 6, 7 and 3, leading to DNA fragmentation and cell death. Oliver et al., “The role of caspases in cell death and differentiation” Drug Resist Updates 2005; 8:163-170; and Iannolo et al., “Apoptosis in normal and cancer stem cells” Crit Rev Oncol Hematol 2008; 66:42-51. The effects of pro-apoptotic signals can be modulated by inhibitors of apoptosis proteins (IAPB), e.g. c-IAP1, c-IAP2, NAIP, Survivin, XIAP, Bruce, ILP-2, and Livin. Nachmias et al., “The inhibitor of apoptosis protein family (IAPs): an emerging therapeutic target in cancer” Semin Cancer Biol 2004; 14:231-243. IAPB directly inhibit caspases and/or catalyze their ubiquitination and proteaseome-mediated degradation. This balance is finely regulated by endogenous inhibitors of IAPB, such as SMAC and HtrA2, which compete with active caspases to bind to IAP. Reed J C., “Drug Insight: cancer therapy strategies based on restoration of endogenous cell death mechanisms” Nature Clin Practice Oncol 2006; 3:388-98. Anti-apoptotic signals such as Bcl-XL can bind and inactivate Apaf-1, and stimulate the release of Smac/DIABLO proteins from the mitochondria, thereby inactivating the IAPs. Qiao et al., “Targeting apoptosis as an approach for gastrointestinal cancer therapy” Drug Resistance Updates 2009; 12:55-64.

The extrinsic pathway of apoptosis is a mechanism by which cells of the immune system trigger apoptosis in ‘unhealthy’ cells through ligand-mediated activation of cell surface death-mediating receptors, such as TNF Receptor 1 (TNFR1), TNF Receptor 2 (TNFR2), CD95/Fas/Apo1, and Death Receptors (tumor necrosis factor-related apoptosis-inducing ligand [TRAIL]-TRAIL receptors) 3-6 (DR3-6). Klein et al., “Killing time for cancer cells” Nat Rev Cancer 2005; 5:573-580; and Degterev et al., “A decade of caspases” Oncogene 2003; 22:8543-8567.

Binding of these receptors by their respective ligands leads to receptor oligomerization and recruitment of death signal adaptor proteins. For example, binding of Fas ligand (Fas-L) to Fas, or TRAIL to TRAIL-R1 leads to recruitment of FADD (Fas-associated death domain), and binding of TNF to TNFR1 leads to recruitment of TRADD (TNFR-associated death domain) lannolo et al., “Apoptosis in normal and cancer stem cells” Crit Rev Oncol Hematol 2008; 66:42-51; and Thorburn et al., “TRAIL receptor-targeted therapeutics: resistance mechanisms and strategies to avoid them” Drug Resist Updates 2008; 11:17-24. The oligomerized receptors and recruited FADD or TRADD form a complex termed DISC (death-inducing signaling complex), which can bind to initiator caspases (caspase 8 and 10), followed by triggering the activation of caspases 7 and 3, and leading to apoptosis.

Recent studies underscore deficiencies in the arbitrary classification of intrinsic and extrinsic apoptosis pathways. First, some signals can activate both pathways, and an extensive crosstalk exists between these two apoptosis pathways. For instance, the transcription factor NF-κβ can activate the transcription of anti-apoptotic genes such as FLIP, Bel-XL, XIAP and cIAP1; however, NF-143 can also enhance the expression of apoptosis-inducing genes such as Fas, Fas-L, TRAIL-R1 and TRAIL-R2. Kucharczak et al., “To be, or not to be: NF-κβ is the answer-role of Rel/NF-κβ in the regulation of apoptosis” Oncogene 2003; 22:8961-8982.

Recent data has further suggested that the extrinsic death-receptor pathway is not limited to cells of the immune system, and that growth control of ‘unhealthy’ cells operates in most tissues containing proliferating cells. Thus, the P2X₇ receptor mechanism controls growth of certain types of epithelial cells, under normal physiological conditions, and, as contemplated herein, impaired P2X₇-mediated apoptosis could contribute to the neoplastic transformation in those tissues. Those discoveries suggest a physiological role for apoptosis in maintaining cellular homeostasis.

The improved understanding of apoptosis has provided a basis for targeted therapies that can induce death of cancer cells or sensitize them to established cytotoxic agents and radiation therapy. Ghobrial et al., “Targeting Apoptosis Pathways in Cancer Therapy” CA Cancer J Clin 2005; 55; 178-194; and Li et al., “Selective anticancer strategies via intervention of the death pathways relevant to cell transformation” Cell Death and Differentiation 2008; 15:1197-1210. Previous reports outlined agents and methods that suggest selective induction of apoptosis in cancer cells might be potentially useful in cancer therapy. Gore et al., “Decitabine” Nat Rev Drug Discov 2006; 5:891-892; and Reu et al., “Overcoming resistance to interferon-induced apoptosis of renal carcinoma and melanoma cells by DNA demethylation” J Clin Oncol 2006; 24:3771-3779; and Gartel A. L., “Transcriptional inhibitors, p53 and apoptosis” Biochim Biophys Acta 2008; 1786:83-86. Such apopotic mechanisms include, but are not limited to, i) activation of the cell surface death receptors Fas, TRAIL and TNF receptors; ii) inhibition of cell survival signaling via EGFR, MAPK and PI3K; iii) altering the balance between pro-apoptotic and anti-apoptotic members of the Bcl-2 family; iv) down-regulating anti-apoptosis proteins such as XIAP, surviving and c-IAP2; e) proteasome inhibitors; f) nonsteroidal anti-inflammatory drugs (NSAIDs) and COX-2 inhibitors; g) peroxisome proliferator-activated receptor (PPAR) ligands; or h) DNA methylation.

Despite the expanse of present research, however, only a small number of therapies directly targeting the apoptotic pathways have advanced into clinical testing, and none have yet achieved approval by the United States Food And Drug Administration. Of the clinical trials that were initiated using agents such as those listed above, many were of limited value because of problems including, but not limited to: i) low efficacy (Cornett et al., “Randomized multicenter trial of hyperthermic isolated limb perfusion with melphalan alone compared with melphalan plus tumor necrosis factor: American College of Surgeons Oncology Group Trial Z0020” J Clin Oncol 2006; 24:4196-4201); ii) toxicity (Jo et al., “Apoptosis induced in normal human hepatocytes by tumor necrosis factor-related apoptosis-inducing ligand” Nat Med 2000; 6:564-567; iii) presence of decoy receptors (DcR1, DcR2, and osteoprotegerin) which bind TRAIL and inhibit apoptosis (Wang et al., “TRAIL and apoptosis induction by TNF-family death receptors” Oncogene 2003; 22:8628-8633; iv) concerns of inducing immunodeficiency with hypogammaglobulinemia; or v) predisposition to develop lymphomas (Rigaud et al., “XIAP deficiency in humans causes an X-linked lymphoproliferative syndrome” Nature 2006; 444:110-114).

B. Apoptosis and Cancer

Defective apoptosis may play a role in the development of cancers. Gasser et al., “The DNA damage response, immunity and cancer” Semin Cancer Biol 2006; 16:344-347; Kujoth et al., “Mitochondrial DNA mutations and apoptosis in mammalian aging” Cancer Res 2006; 66:7386-7389; and Rodriguez-Nieto et al., “Role of alterations in the apoptotic machinery in sensitivity of cancer cells to treatment” Curr Pharm Des 2006; 12:4411-4425. In fact, one of the hallmarks of cancer is the development of mechanisms that evade apoptosis, and the loss of pro-apoptotic signals and gain of anti-apoptotic mechanisms contribute to tumorigenesis and the cancer phenotype. Thus, defective apoptotic mechanisms allow genetically unstable cancer cells to avoid elimination and confer resistance to cancer treatments. Hanahan et al., “The hallmarks of cancer” Cell 2000; 100:57-70; and Cummings et al., “Apoptosis pathway-targeted drugs: from the bench to the clinic” Biochim Biophys Acta 2004; 1705:53-66.

Since apoptosis does not elicit inflammatory or immune response, this type of cell death is the preferred way of cancer cell killing by various treatments. The selective induction of apoptosis in cancer cells is emerging as a promising therapeutic approach for many cancers, and modulating the apoptotic pathways may be involved in mechanisms including, but not limited to, i) inducing tumor-cell death; ii) increasing responses to chemotherapy, radiotherapy and other targeted therapies; or iii) prevention of the neoplastic transformation. Ziegler et al., “Therapeutic targeting of apoptosis pathways in cancer” Curr Opin Oncol 2008; 20:97-103.

Levels of the functional P2X₇ receptor in cancer epithelial cells of the ectoderm, the uro-genital sinus, and the distal paramesonephric duct are reported to be lower compared to normal cells (infra). The lesser expression of the P2X₇-receptor could be the result of the neoplastic transformation. Thus, in endometrial and bladder cells low expression of the P2X₇ receptor was found already in pre-cancerous and early cancerous cells, but not in hyperplastic benign cells. Li et al., “The P2X7 Receptor: A novel biomarker of uterine epithelial cancers” Cancer Epidemiol Biomarkers Preven 2006; 15:1-8; Li et al., “P2X7 receptor expression is decreased in epithelial cancer cells of ectodermal, uro-genital sinus, and distal paramesonephric-duct origin” Purinergic Signal 2009; 5:351-368; and Li et al., “Decreased expression of P2X7 in endometrial epithelial pre-cancerous and cancer cells” Gynecol Oncology 2007; 106:233-243. As the data presented herein demonstrates, the carcinogenic process could have induced reduced expression of the P2X₇ at early stages of cancer development. Alternatively, the neoplastic transformation could have been triggered preferentially in cells expressing low levels of the receptor. This possibility is supported by data in uterine cervical epithelia, where low expression of the P2X₇ receptor was found already in dysplastic (precancerous) cells. Few cases of dysplasia progress to cancer, so it is possible that low expression of the receptor preceded the neoplastic transformation. Song et al., “Risk factors for the progression or persistence of untreated mild dysplasia of the uterine cervix” Int J Gynecol Cancer 2006; 16:1608-1613. Accordingly, cells harboring defective P2X₇ expression mechanism have escaped apoptosis, and were rendered susceptible to carcinogenic stimuli and the neoplastic transformation.

In both scenarios, low expression of the P2X₇ receptor could promote cancer development, because decreased apoptosis due to reduced receptor expression can facilitate the growth of neoplastic cells. A recent study tested the hypothesis that in tissues at risk for undergoing malignant transformation augmentation of P2X₇-mediated apoptosis could inhibit cancer development. Fu et al., “Activation of P2X(7)-mediated apoptosis Inhibits DMBA/TPA-induced formation of skin papillomas and cancer in mice” BMC Cancer 2009; 9:114

### III. P2X Receptor Family

The human P2X₇ receptor gene is localized to chromosome 12q24 and comprises 13 exons. Buell et al., “Gene structure and chromosomal localization of the human P2X₇ receptor” Receptors Channels 5:347-354 (1998). Some genetic mutations in the P2X₇ receptor gene have been described, but none regarding cervical cancer. Feng et al., “A truncated P2X₇ receptor variant (P2X₇₋ⱼ) endogenously expressed in cervical cancer cells antagonizes the full-length P2X₇ receptor through hetero-oligomerization” J Biol Chem. 281:17228-17237 (2006). Since the overall prevalence of known chromosomal abnormalities in cervical cancers is low, genetic mutations cannot be considered the main etiological factor of the disease.

It has been reported that the P2X₇ receptor may belong to the P2X sub-family of P2 nucleotide receptors which are membrane-bound, ligand-operated channels. Buell et al, “P2X receptors: an emerging channel family” Eur J Neurosci 8:2221-2228 (1996); Soto et al, “Cloned ligand-gated channels activated by extracellular ATP (P2X receptors)” J Membr Biol 160:91-100 (1997); Dubyak et al., “Signal transduction via P2-purinergic receptors for extracellular ATP and other nucleotides” Am J Physiol 265:C577-C606 (1993); Ralevic et al., “Receptors for purines and pyrimidines” Pharmacol Rev 50:413-492 (1998); and Khakh et al, “Current status of the nomenclature and properties of P2X receptors and their subunits” Pharmacol Rev 53: 107-118 (2001). For example the nucleotide, adenosine triphosphate (ATP), is believed to be a naturally occurring P2X₇ receptor ligand. ATP has been reported to be constitutively secreted by cells wherein ATP levels in extracellular fluids may be present in a low micromolar range. Sperlagh et al, “ATP released by LPS increases nitric oxide production in raw 264.7 macrophage cell line via P2Z/P2X7 receptors” Neurochem Int 33:209-215 (1998); Grahames et al, “Pharmacological characterization of ATP- and LPS-induced IL-1β release in human monocytes” Br J Pharmacol 127: 1915-1921 (1999); Henriksen et al., “Effect of ATP on intracellular pH in pancreatic ducts involves P2X7 receptors” Cell Physiol Biochem 13:93-102 (2003); Loomis et al, “Hypertonic stress increases T-cell Interleukin-2 expression through a mechanism that involves ATP release, P2 Receptor, and p38 MAPK activation” J Biol Chem 278:4590-4596 (2003); and Wang et al, “P2X7-receptor mediated apoptosis of human cervical epithelial cells” Am J Physiol 287:C1349-C1358 (2004). Early studies suggested that, in contrast to other types of ATP receptors, activation of the P2X₇ receptor might require a relatively high concentration of ligand. Ralevic et al., “Receptors for purines and pyrimidines” Pharmacol Rev 1998, 50:413-492. However, the data shown herein demonstrate that a threshold effect of P2X₇-mediated apoptosis occurs at nanomolar concentrations of ATP, suggesting that ATP levels which are present in the extracellular fluid are sufficient to activate the P2X₇ receptor.

One cellular effect of P2X₇ receptor activation may involve the formation of pores in the plasma membrane. Wang et al., “Anti-apoptotic effects of estrogen in normal and in cancer human cervical epithelial cells” Endocrinology 2004, 145:5568-5579. For example, in uterine epithelial cells, formation of P2X₇ receptor pores induces apoptosis by a mechanism believed to involve influx of Ca²⁺ via the P2X₇-pores in parallel with an activation of the mitochondrial caspase-9 pathway. North R A, “Molecular physiology of P2X receptors” Physiol Rev 2002, 82:1013-1067; Wang et al., “Anti-apoptotic effects of estrogen in normal and in cancer human cervical epithelial cells” Endocrinology 2004, 145:5568-5579; and Feng et al., “A truncated P2X₇ receptor variant (P2X₇₋ⱼ) endogenously expressed in cervical cancer cells antagonizes the full-length P2X₇ receptor through hetero-oligomerization” J Biol Chem 2006, 281:17228-17237. P2X₇ receptor activation by a brief exposure to extracellular ATP has been reported to open cation channels that apparently allow Ca²⁺, Na⁺ and K⁺ influx. Surprenant et al, 1996. Further, a longer exposure to ATP may induce pore formation in the plasma membrane. Virginio et al, 1999.

The P2X₇ receptor is believed to play a role in cell growth because the receptor is expressed by proliferating cells. Li et al., “The P2X₇ Receptor: A novel biomarker of uterine epithelial cancers” Cancer Epidemiol Biomarkers Preven 2006, 15:1-8. Further, it has been reported that activation of the P2X₇ receptor induces apoptosis thereby having a regulatory impact on cell growth. Wang et al., “EGF facilitates epinephrine inhibition of P2X₇-receptor mediated pore formation and apoptosis: a novel signaling network” Endocrinology 2005, 146:164-174.

Until recently, relatively little was known about the in vivo biological role of the P2X₇ receptor. Earlier studies suggested involvement of the P2X₇ receptor in inflammatory and immune processes since the receptor is expressed in the islets of Langerhans and inflammatory dendritic epidermal cells and in cultured immature dendritic epidermal cells. Georgiou et al., “Human epideinial and monocyte-derived langerhans cells express functional P2X receptors” J Invest Dermatol 125:482-490 (2005); and Mutini et al, “Mouse dendritic cells express the P2X7 purinergic receptor: Characterization and possible participation in antigen presentation” J Immunol 163:1958-1965 (1999). Overexpression of P2X₇ was found in lesional skin of psoriasis and atopic dermatitis, where an intense P2X₇ immunoreactivity was confined to the cell membrane of the basal layer. P2X₇ has been suggested to play a role in chemokine secretion by normal keratinocytes but available data are inconsistent. For example, one study reported that the treatment of cultured normal keratinocytes with the P2X₇ specific agonist 2′,3′-0-(4-benzoylbenzoyl)-adenosine 5′-triphosphate (BzBzATP) increased IL-6 release, while a second report found that BzBzATP decreased chemokine secretion. Inoue et al, “Extracellular ATP has stimulatory effects on the expression and release of IL-6 via purinergic receptors in normal human epidermal keratinocytes” J Invest Dermatol 127:362-371 (2007); and Pastore et al, “Stimulation of purinergic receptors modulates chemokine expression in human keratinocytes” J Invest Dermatol 127:660-667 (2007).

Studies have also suggested a role for P2X₇ in the control of epidermal growth, but most studies were observational. The P2X₇ receptor expression has been found in: i) normal tissues; ii) precancerous epidermal tissues; and skin cancer cells. P2X₇ receptor immunoreactivity was found throughout the epidermis, including in the basal/parabasal germinative regions of the epidermis. P2X₇ receptors were detected as early as 8-11 weeks in human fetal epidermis cells (i.e., for example, periderm), wherein the receptors co-localized with caspase-3 and TUNEL staining. Co-localization of the P2X₇ receptors with such apoptosis-related markers was also reported in adult human epidermis, and recent studies reported BzBzATP-induced cell death in normal and cancer keratinocytes. Greig et al., (2003) “Purinergic receptors are part of a functional signaling system for proliferation and differentiation of human epidermal keratinocytes” J Invest Dermatol 120:1007-1015; Greig et al., (2003) “Expression of purinergic receptors in non-melanoma skin cancers and their functional roles in A431 cells” J Invest Dermatol 121:315-327; Greig et al., (2003) “Purinergic receptors are part of a signaling system for keratinocyte proliferation, differentiation, and apoptosis in human fetal epidermis” J Invest Dermatol 121:1145-1149; Slater et al., “Differentiating keratoacanthoma from squamous cell carcinoma by the use of apoptotic and cell adhesion markers” Histopathology 47:170-178 (2005); White et al, “Human melanomas express functional P2X7 receptors” Cell Tissue Res 321:411-418 (2005); and Pastore et al, “Stimulation of purinergic receptors modulates chemokine expression in human keratinocytes” J Invest Dermatol 127:660-667 (2007).

Although these existing observational data suggest that the P2X₇ may regulate growth of epithelial cells little is known about the biological role of the P2X₇ receptor in the epidermal layers. For example, no previous studies have investigated experimentally the biological role of the P2X₇ receptor in vivo. The data presented herein demonstrates that P2X₇ receptors have an in vivo physiological role in the control of growth of epidermal epithelial cells. The data also suggest that this growth control may occur through apoptotic mechanisms, and that pharmacological stimulation of the receptor could inhibit development of epidermal neoplasia. For example, the presented data collected in cultured human normal keratinocytes and/or cancer keratinocytes provide direct evidence that P2X₇ receptors control the growth of cells through regulation of apoptosis. Specifically, in vivo mouse data discussed below show that locally applied P2X₇ receptor agonists inhibit DMBA/TPA-induced papilloma formation.

The translated product of the human P2X₇ transcript is a 595 aa linear polypeptide, predicted to traverse the plasma membrane and to possess two intracellular domains and an extracellular domain with the following topology. The P2X₇ polypeptide may comprise the following regions:

a) N-terminus (aa 1-25), which forms intracellular complexes with several proteins including β2 integrin, receptor-like tyrosine phosphatase (RPTP), α-actin, phosphatidylinositol 4-kinase, membrane-associated guanylate kinase, and several heat shock proteins. Kim et al., “Proteomic and functional evidence for a P2X7 receptor signalling complex” EMBO J 20:6347-6358 (2001). These complexes may mediate some P2X₇-dependent signaling

b) The first transmembrane segment (aa 26-46) c) Extracellular domain (aa 47-334), which contains the ligand binding site [46-49], and five putative N glycosylation sites [43], of which Asn187, Asn213, and Asn241 are required to confer functionality. Buell et al., “Gene structure and chromosomal localization of the human P2X7 receptor” Receptors Channels 5:347-354 (1998); and Feng et al., “A truncated P2X7 receptor variant (P2X7-j) endogenously expressed in cervical cancer cells antagonizes the full-length P2X7 receptor through hetero-oligomerization” J Biol Chem 281:17228-17237 (2006).

d) The second transmembrane segment (aa 335-355).

e) A long C-terminus (aa 356-595) that is required for pore formation. Domains within the carboxy-terminal tail of the P2X₇ also direct trafficking and stabilize expression of the receptor in the plasma membrane. Buell et al., “Gene structure and chromosomal localization of the human P2X7 receptor” Receptors Channels 5:347-354 (1998); Feng et al., “A truncated P2X7 receptor variant (P2X7-j) endogenously expressed in cervical cancer cells antagonizes the full-length P2X7 receptor through hetero-oligomerization” J Biol Chem 281:17228-17237 (2006); Boldt et al., “Glu496Ala polymorphism of human P2X7 receptor does not affect its electrophysiological phenotype” Am J Physiol 284:C749-56 (2003); Surprenant et al., “The cytolytic P2Z receptor for extracellular ATP identified as a P2X receptor (P2X7)” Science 272:735-738 (1996); Denlinger et al., “Mutation of a dibasic amino acid motif within the C-terminus of the P2X7 nucleotide receptor results in trafficking defects and impaired function” J Immunol 171:1304-1311 (2003); and Cheewatrakoolpong et al., “Identification and characterization of splice variants of the human P2X7 ATP channel” Biochem Biophys Res Comm 332:17-27 (2005).

### IV. In Vivo BzBzATP On Cancerous Tissue Development

Experiments were performed to test the hypothesis that activation of the P2X₇ receptor could inhibit development of epidermal neoplasia. These experiments utilized the mouse two-step DMBA/TPA skin neoplasia model, which involves tumor initiation by local treatment with DMBA, followed by tumor promotion by local treatment with TPA. Agarwal et al, “Inhibitory effect of 18beta-glycyrrhetinic acid on 12-O-tetradecanoyl phorbol-13-acetate-induced cutaneous oxidative, stress and tumor promotion in mice” Redox Rep 10:151-157 (2005); and Guo et al, “Disruption of EphA2 receptor tyrosine kinase leads to increased susceptibility to carcinogenesis in mouse skin” Cancer Res 66:7050-7058 (2006). Mice had their dorsal skin shaved, and DMBA was applied once by topical application onto the shaved dorsal skin. TPA treatment by topical application onto the shaved dorsal skin was started one week later and continued twice a week for 12 weeks.

Epithelial cancers usually develop from a premalignant lesions, e.g., a papilloma, and the cancer risk of premalignant epithelial lesions may vary from 0.1% to 20%. 48. Reibel J, “Prognosis of oral pre-malignant lesions: Significance of clinical, histopathological, and molecular biological characteristics” Crit Rev Oral Biol Med 2003, 14:47-62; Fu et al., “The actinic (solar) keratosis: A 21st century perspective” Arch Dermatol 2003, 139:66-70; and Lindeque B G, “Management of cervical premalignant lesions” Best Pract Res Clin Obstet Gynaecol 2005, 19:545-561.

Animals were divided into three groups: Control mice (n=15) that had their back shaved and were treated only with the vehicle; DMBA/TPA-treated mice (n=15); and DMBA/TPA-treated mice that were co-treated with BzBzATP twice a week from week −2 (i.e. two weeks prior to DMBA) (n=14). All treatments were applied locally on the shaved dorsal skin. Papilloma development was monitored weekly from week 5 to 12 after the DMBA. Endpoints were percent animals with at least one papilloma; number of papilloma per animal; and mean papilloma size (e.g., millimeters of the largest lesion dimension). None of the animals in the control group had developed papillomas.

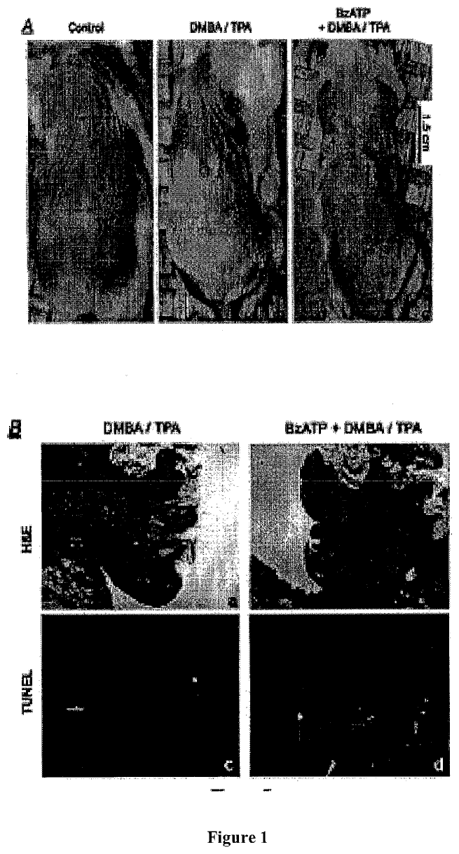

Fourteen (14) out of the fifteen (15) animals in the DMBA/TPA group (93%) and twelve (12) out of the fourteen (14) (78%) animals in the DMBA/TPA+BzBzATP group developed at least one skin papilloma at week 12. See, FIG. 1A. Using time-to-event data (i.e., for example, a Kaplan-Meier analysis for “papilloma-free” states) the log-rank test between the DMBA/TPA and DMBA/TPA+BzBzATP groups was not significant (p=0.273). However, analysis of the proportion having a papilloma at weeks 5-12 separately gave a borderline (p=0.055) result at week 6 of treatment. Specifically there were 13/15 (86.7%) in the DMBA/TPA group versus 7/13 (53.8%) in the DMBA/TPA+BzBzATP group having at least one papilloma.

In the DMBA/TPA and DMBA/TPA+BzBzATP groups the mean number of papillomas per animal increased over the 12 week study period, but the increase in papillomas in the DMBA/TPA+BzBzATP group was smaller than in the DMBA/TPA group. See, FIG. 1B. An independent samples t-test for weeks 5-12 for the DMBA/TPA and DMBA/TPA+BzBzATP groups revealed borderline significant difference at weeks 8 and 9 (p=0.051, 0.057) and a to significant difference at week 10 (2.3±0.34 and 1.23±0.34 papillomas per animal (mean±SEM, respectively, p=0.033). Repeated measures analysis of variance (ANOVA) yielded a significant time effect (p<0.01), a borderline group effect (p=0.067) and a non-significant time*group interaction effect (p=0.290), for the DMBA/TPA and DMBA/TPA+BzBzATP curves. The latter indicates parallel non-interacting trends for the DMBA/TPA and DMBA/TPA+BzBzATP curves.

In the DMBA/TPA and DMBA/TPA+BzBzATP groups, the mean papilloma size per animal increased over the 12 weeks study period, but the increase in the DMBA/TPA+BzBzATP group was smaller than in the DMBA/TPA group. See, FIG. 1C. An independent samples t-test for weeks 5-12 for the DMBA/TPA and DMBA/TPA+BzBzATP groups revealed significant differences at all weeks for the mean size (with respective p values ranging from 0.005 to 0.029). Thus, for example, at week 12 mean papilloma size (mm) per animal was 5.86±0.91 versus 3.46±0.73 (mean±SEM), respectively (p=0.01). Likewise, repeated measures ANOVA yielded a significant time effect (p<0.01), a significant group effect (p=0.011) and a non-significant time*group interaction effect (p=0.113), for the DMBA/TPA and DMBA/TPA+BzBzATP curves. The latter indicates, again, parallel non-interacting trends for the DMBA/TPA and DMBA/TPA+BzBzATP curves.

Interestingly, papillomas induced by DMBA/TPA treatment in mice co-treated with BzBzATP were less hypertrophic and displayed less frequently ulceration and necrosis. See, FIG. 1A(c). Also, in these mice the formed papillomas frequently showed various degrees of involution. See, FIG. 1A(c), arrows.

Collectively, these data show that in DMBA/TPA-treated mice, co-treatment with BzBzATP applied locally on the skin tended to decrease the incidence of papilloma formation; it decreased the mean number of papillomas per animal by about 25% and the mean papilloma size by about 45%. In addition, in mice co-treated with BzBzATP, formed papillomas underwent more frequently involution.

Large papillomas (i.e., for example, greater than 5 mm in diameter) were biopsied at week 10 from one animal of each of the two treatment groups. These tissues were assayed for microscopic H&E evaluation, Ki67 immunostaining, and TUNEL. There were no differences among the two groups in tissue architecture or histology or Ki67 immunoreactivity. See, FIG. 1B(a,b); and not shown, respectively. Papilloma tissues from animals in the DMBA/TPA group showed weak TUNEL staining. See, FIG. 1B(c). In contrast, papilloma tissues from animals in the DMBA/TPA+BzBzATP group showed intense TUNEL staining in basal/parabasal regions of the papilloma epithelial regions. See, FIG. 1B(d), arrow).

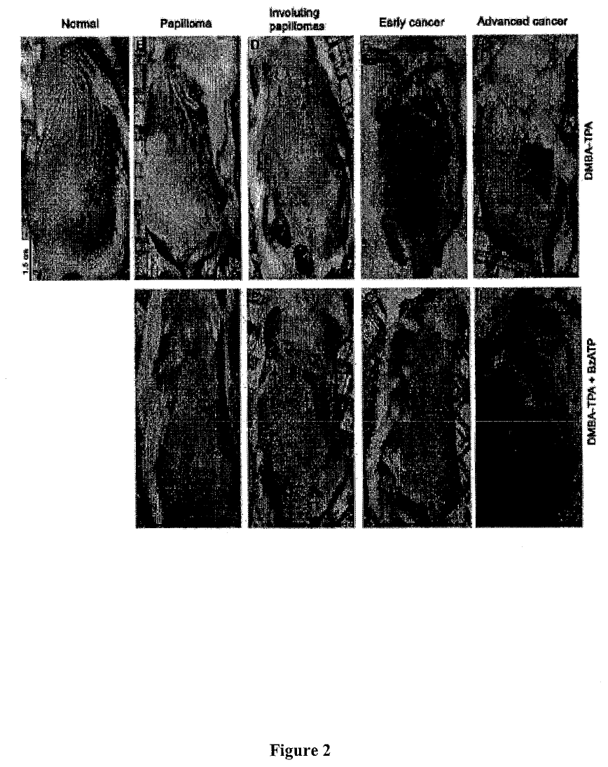

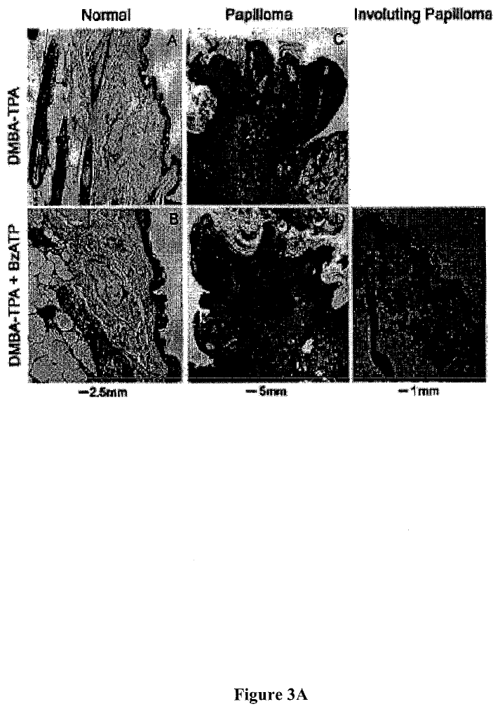

After twenty-eight (28) weeks, lesions induced by the local administration of DMBA/TPA progressed into formation of squamous spindle-cell carcinomas. See, FIGS. 2 and 3. As the data presented show, about one-third of the papillomas involuted after week 14 and the remaining persisted either as non-cancerous papillomas, or transformed to cancerous lesions. All cancerous lesions arose from pre-existing papillomas, while none of the animals in the control group had developed skin lesions. See, FIG. 2A. There were no significant differences in the morphological or histological characteristics of the unaffected normal skin in the DMBA/TPA and the DMBA/TPA+BzBzATP groups. See, FIGS. 2A-I and FIGS. 3A-B, respectively. Similarly, there were no significant differences in the morphological and histological characteristics of papillomas in the DMBA/TPA and DMBA/TPA+BzBzATP groups. See, FIGS. 2B-E and FIGS. 3C-D, respectively.

After week 14, some papillomas remained intact while other started to involute in both the DMBA/TPA and DMBA/TPA+BzBzATP groups. See, FIGS. 2D-E, and FIG. 3E. However, in both groups most papillomas (i.e., for example, about two-thirds) underwent cancerous transformation to squamous cell carcinomas with spindle-cell changes. See, Figures. 3F-I. There were no significant changes in the morphological and histological characteristics of cancers in the two groups. See, FIGS. 2F-I and not shown, respectively.

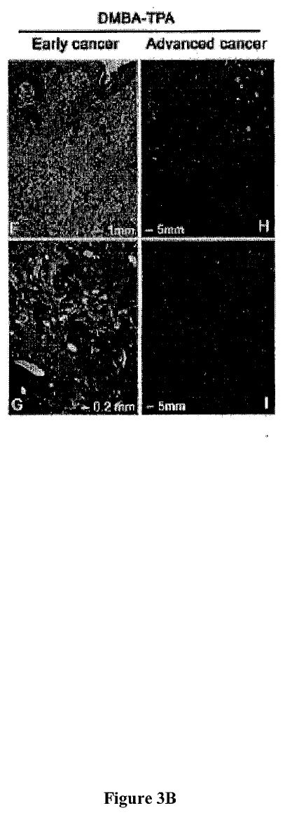

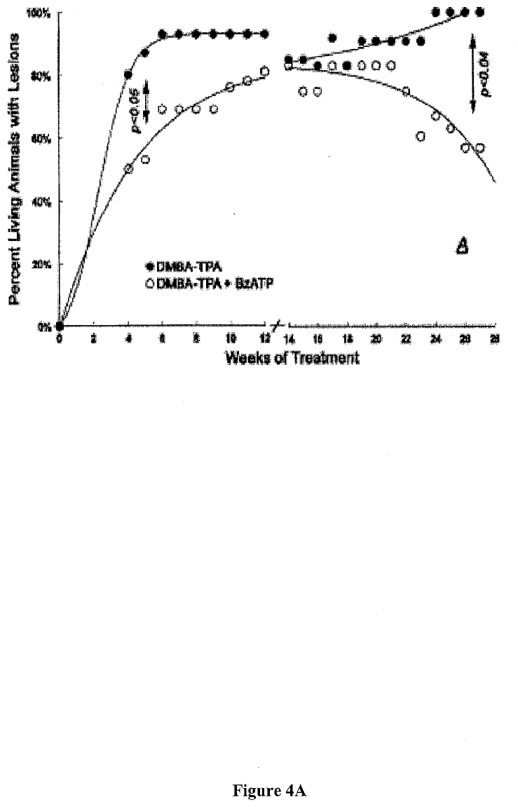

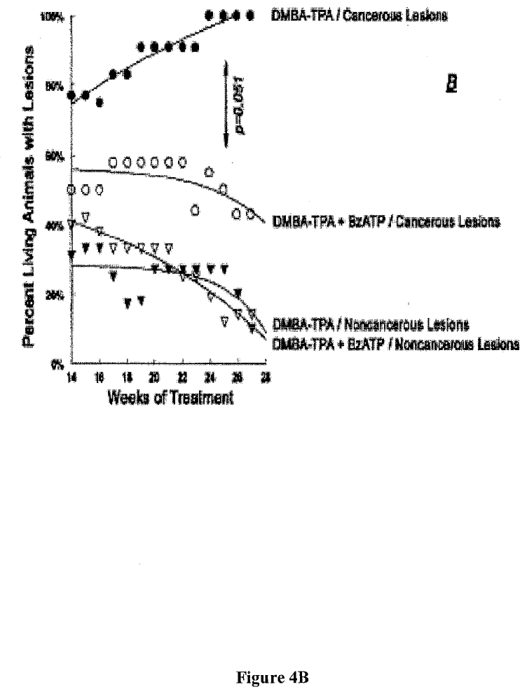

Overall, the data show that co-treatment with BzBzATP, applied locally on skin areas exposed to DMBA/TPA altered the incidence and pattern of skin lesions having progression to skin cancer. To evaluate the effects of BzBzATP, changes in skin lesions in the DMBA/TPA and DMBA/TPA+BzBzATP groups were compared relative to the length of treatment. Since formation of papillomas and cancerous lesions was time-related (i.e., for example, a marked cut-off occurs between weeks 13-14; see, FIG. 4A), data were analyzed separately for weeks 0-12 and weeks 14-28.

During weeks 0-12, the proportion of living animals with papillomas tended to be lower in the DMBA/TPA+BzBzATP group than in the DMBA/TPA group, and analysis of the proportion of living animals having a papilloma separately was significant at week 5 of treatment ((p<0.05): 48±12% versus 80±10%, respectively). The proportion of living animals with any skin lesion between weeks 14-21 was similar in the two groups. Nonetheless, the proportion of living animals with any skin lesion differed significantly among the groups in weeks 22-28. See, FIG. 4A.

During the 28 week timeperiod, the proportion of living animals with non-cancerous lesions (i.e., for example, existing and involuting papillomas) decreased in both groups. In contrast, the proportion of living animals with cancerous lesions in the DMBA/TPA group increased steadily, while in the DMBA/TPA+BzBzATP group the proportion of living animals with cancerous lesions decreased over time. For example, in week 28 the proportions of living animals with cancerous lesions in the DMBA/TPA and the DMBA/TPA+BzBzATP groups were 100% and 43±9%, respectively. See, FIG. 4B.

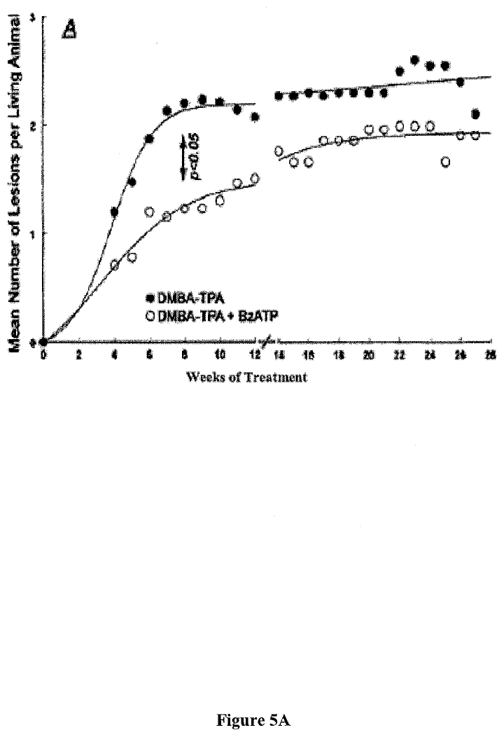

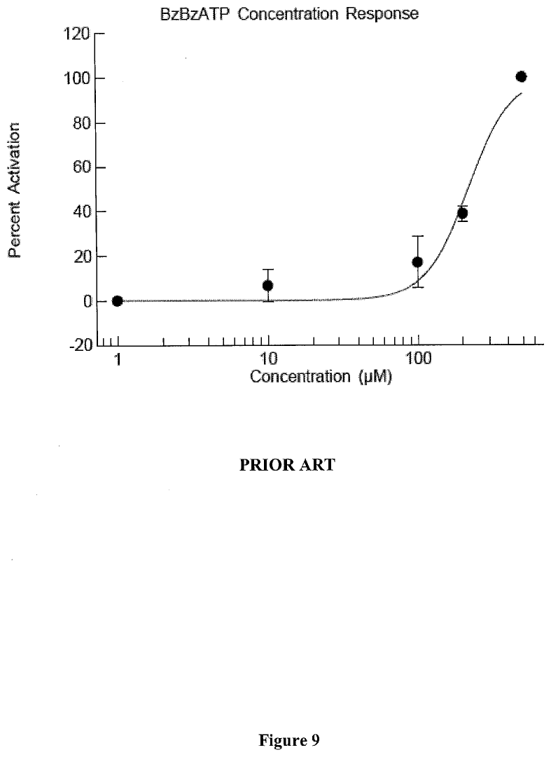

In both groups, the mean number of papillomas per living animal increased between weeks 0-12, but the increase in the DMBA/TPA+BzBzATP group tended to be smaller than in the DMBA/TPA group. See, FIG. 5A. An independent samples t-test revealed a significant difference at week 10 (i.e., for example, 2.3±0.5 and 1.2±0.4 papillomas per animal (mean±SD, respectively, p<0.04)). Also, a repeated measures analysis of variance yielded a significant time effect for the DMBA/TPA and DMBA/TPA+BzBzATP curves between weeks 0-12 (p<0.01). See, FIG. 5A. Between weeks 14-28, the mean number of total lesions per living animal was not significantly different between the two groups. See, FIG. 5A. In both groups, the mean number of non-cancerous lesions decreased over the 14-28 weeks period. See, FIG. 5B. The mean number of cancerous lesions, however, remained the same. See, FIG. 9C.

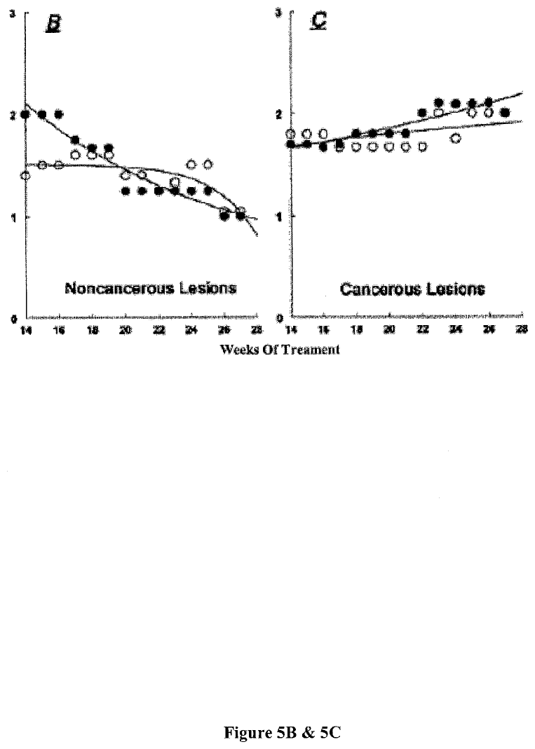

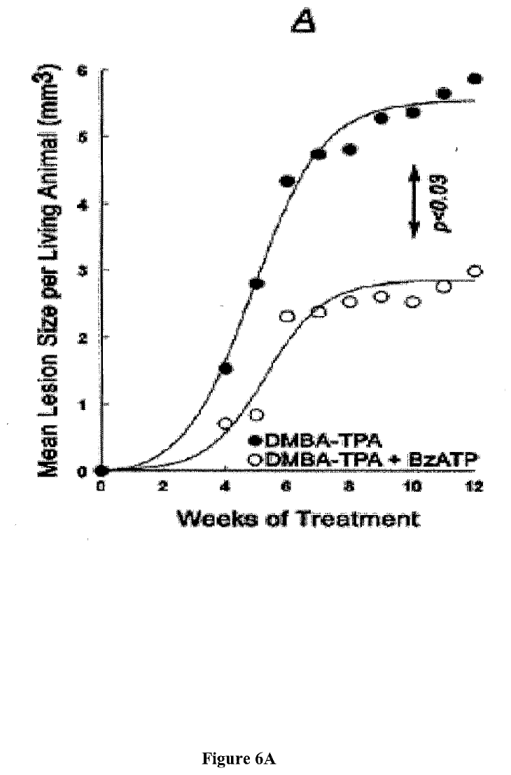

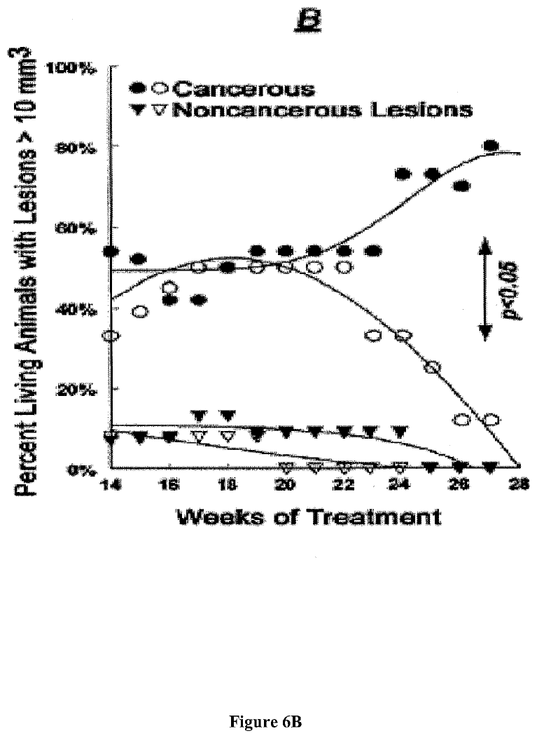

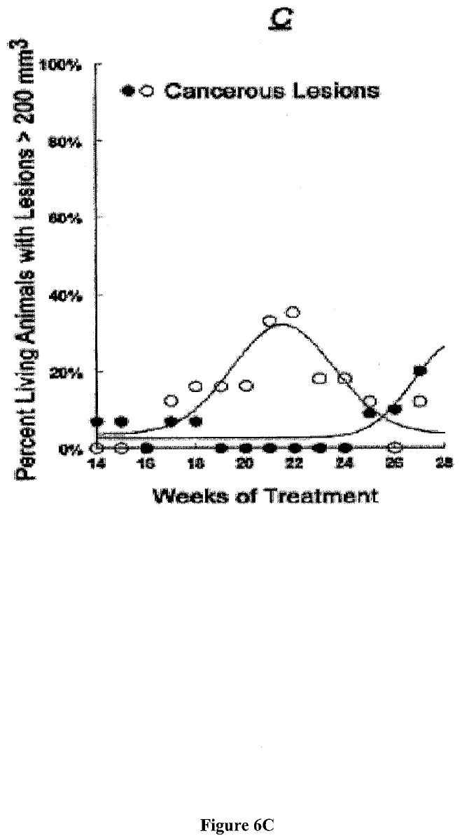

Animals in both groups were compared relative to the total size of lesions (in mm³) per animal. In both groups, the mean total papillomas size per living animal increased between weeks 0-12, but the increase in the DMBA/TPA+BzBzATP group was smaller than in the DMBA/TPA group. See, FIGS. 2B, 2C, and 6A. An independent samples t-test revealed significant differences at all weeks for mean total papillomas size (p<0.01-0.03). See, FIG. 6A. For example, in week 12 mean total papillomas size (in mm) per animal was 5.8±1.1 versus 3.4±1.0 (mean±SD), respectively (p<0.01). Likewise, a repeated measures analysis of variance yielded a significant time effect (p<0.01); a significant group effect (p<0.02); and a non-significant time*group interaction effect (p>0.1), for both DMBA/TPA and DMBA/TPA+BzBzATP data sets. The non-significant time*group interaction term, indicates non-interacting trends between the DMBA/TPA and DMBA/TPA+BzBzATP groups.

Between weeks 14-28, the variability of the lesion sizes among the two groups was large. Although it is not necessary to understand the mechanism of an invention, it is believed that this variability was due to an unproportional excessive growth of some lesions relative to others. See, FIGS. 2D-2I. This precluded a comparison of the means of lesion size among the two groups. However, since most non-cancerous lesions in both groups tended to be smaller than 10 mm and the proportion of animals with non-cancerous lesions of >10 mm was low in both groups (i.e., for example, <10%). See, FIG. 6B, triangles. Data representing the proportion of living animals with cancerous lesions >10 mm were compared among the two groups. After week 28, a significantly smaller proportion of living animals was observed in the DMBA/TPA+BzBzATP group with cancerous lesions >10 mm than in the DMBA/TPA group. For example, in week 28 the proportion of living animals with cancerous lesions >10 mm³ were 81%±8% as compared to 16±4% in the DMBA/TPA and the DMBAJTPA+BzBzATP groups, respectively. See, FIG. 6B.

Five (5) mice in the DMBA/TPA+BzBzATP group survived despite having developed relatively large cancerous lesions, while maintaining normal weight and exhibiting normal behavior. See, FIG. 2I. In contrast, most mice in the DMBA/TPA group with smaller cancerous lesions met IACUC euthanization requirements due to poor general condition and excessive tumor burden. See, FIG. 2H. Analysis of the proportion of living animals with cancerous lesions >200 mm³ showed a tendency for higher proportion of animals in the DMBA/TPA+BzBzATP group than in the DMBA/TPA group, but the differences did not reach statistical significance. See, FIG. 6C.

V. Non-BzBzATP Derivatives have Improved P2X₇ Efficacy

In some embodiments, the present invention contemplates compositions and methods comprising non-BzBzATP derivatives (ATPd), wherein the ATPd's have improved P2X7 efficacy in comparison to BzBzATP. In one embodiment, the present invention contemplates an ATPd including, but not limited to, benzoyl-ATP, cinnamoyl-ATP, 3-phenoxybenzoyl-ATP lauroyl-ATP and/or acetate-ATP.

In some embodiments, the ATPd's comprise a 3-O-ribose modified ATP. Although it is not necessary to understand the mechanism of an invention, it is believed that since ATP is an active P2X7 agonist (e.g, EC₅₀˜100 μM) and BzBz-ATP is also an active P2X7 agonist (e.g., EC₅₀˜50 μM), then the 3,5 BzBz derivative does not a critical substitutent to promote P2X7 agonist activity and can be replaced by any organic radical. In some embodiments, the present invention contemplates the design and synthesis of ATPd's wherein: i) the 3-O-ribose comprises an organic acid substituent (e.g., for example, with as a monoester); ii) the organic acid substituent is ‘generally recognized as safe’ (GRAS); iii) the organic acid substituent imparts sufficient hydrophobicity such that the log P ranges between approximately 2-6 (e.g., favoring tissues penetration and/or absorption); iv) the organic acid substitutent comprises an aliphatic derivative; v) the organic acid substitutent comprises an aromatic derivative; and vi) the organic acid substitutent comprises a low ultraviolent (UV) radical generation activity. For example, lauroyl-ATP, benzoyl-ATP and cinnamoyl-ATP all have low or no UV radical generation activity, whereas the previously reported BzBz-ATP compound has a high degree of UV radical generation activity.

Electrochemical patch clamp assay performed in accordance with Example II demonstrated that ATPd's resulted in greater calcium flux than the previously reported BzBzATP compound. These results demonstrate that the ATPds have improved P2X7 receptor activation efficacy than BzBzATP. The in vitro effects of three exemplary ATPds on cloned P2X7 ATP-activated channel (human P2RX7 gene expressed in HEK293 cells) were evaluated in agonist mode on the QPatch HT® (Sophion Bioscience A/S, Denmark), an automatic parallel patch clamp system.

These test compounds were evaluated at 1, 10, 100 and 500 μM with each concentration tested in at least three cells (n≥3). 3′-O-(benzoyl)-adenosine-5-triphosphate was tested twice with two different cell passages (passages 52 and 36). All other ATPds were tested using passage 36. Each recording involved three applications of the vehicle control (HBPS), three applications each of four concentrations of the ATPd and ended with three applications of 200 μM benzoylbenzoyl-ATP (BzBzATP, Sigma Cat. B6396). Each application lasted for a period of five seconds followed by a 25 second washout period. Current amplitude was determined using the average of three ATPd applications with the average vehicle control subtracted. The % activation was calculated using the vehicle control as a baseline and 200 μM BzBzATP as the maximum activation. Only cells with stable BzBzATP values were included in the analysis

Electrochemical patch clamp tests using either ATP or BzBz-ATP were compared to testing performed with the ATPds. Concentration response data were fit to an equation of the following form:

% Activation={1−1/[1+([Test]/EC50)N]}100%

where [Test] is the concentration of an ATPd, EC₅₀ is the concentration of an ATPd producing half-maximal activation, N is the Hill coefficient and % activation is the percentage of ion channel current activated at each concentration of an ATPd. Nonlinear least squares fits were solved with the XLfit add-in for Excel 2003 (Microsoft, Redmond, Wash.). EC₅₀ values obtained are specific to this test and may differ from values obtained by other methods. Measurements are performed in triplicate in order to reduce variability. Data and results should be taken as indicative, not quantitative and interpretations made relative to the positive control (e.g., for example ATP and/or BzBzATP). A three-fold decrease in EC₅₀ is an indication of potential increased potency, but the factor (3×) itself is not precise.

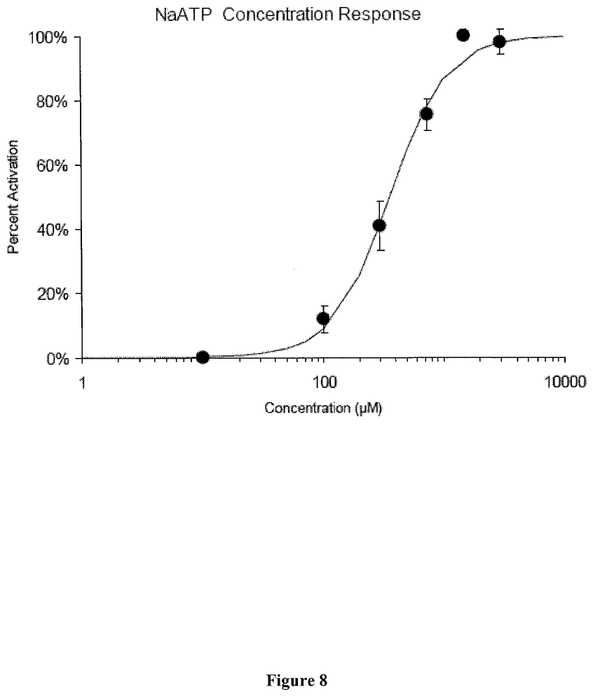

As ATP is the natural ligand for the P2X7 receptor it was of interest to determine the receptor activation characteristics in the native state. See, Table 1 and FIG. 8.

TABLE 1
P2X7 Receptor Activation Characteristics Using ATP sodium salt.

| | | | Mean % | | | |
|---|---|---|---|---|---|---|
| Test Article | EC₅₀ | Conc | P2X7 | Standard | Standard | |
| ID | (μM) | (μM) | Activation | Deviation | Error | n |
| NaATP | 362.634 | 0 | −15.5 | 21.4 | 6.8 | 10 |
| | | 10 | 0.0 | 0.0 | 0.0 | 10 |
| | | 100 | 11.8 | 13.3 | 4.2 | 10 |
| | | 300 | 40.9 | 24.6 | 7.8 | 10 |
| | | 750 | 75.4 | 15.5 | 4.9 | 10 |
| | | 1500 | 100.0 | 0.0 | 0.0 | 10 |
| | | 3000 | 98.1 | 11.5 | 3.6 | 10 |

For additional comparative purposes a previously reported P2X7 receptor agonist (BzBz-ATP) as also tested to determine P2X7 receptor activation characteristics. See, Table 2 and FIG. 9.

TABLE 2
P2X7 Receptor Activation Characteristics Using BzBz-ATP.

| | | | | | | | | Individual | |
|---|---|---|---|---|---|---|---|---|---|
| Test | | | | Mean % | | | | Data | Current |
| Article | | EC₅₀ | Conc | P2X7 | Standard | Standard | | Points (% | Amplitude |
| ID | Lot | (μM) | (μM) | Activation | Deviation | Error | n | Activation) | (pA) |
| BzBzATP | 050M5050V | 218.260 | 1 | 0.0 | 0.2 | 0.1 | 3 | 0.0 | −0.2 |
| | | | | | | | | −0.2 | 0.2 |
| | | | | | | | | 0.1 | −1.5 |
| | | | 10 | 6.7 | 7.2 | 4.1 | 3 | 2.2 | −21.6 |
| | | | | | | | | 14.9 | −13.0 |
| | | | | | | | | 2.9 | −52.5 |
| | | | 100 | 16.9 | 11.5 | 6.7 | 3 | 7.2 | −71.7 |
| | | | | | | | | 29.6 | −25.8 |
| | | | | | | | | 13.8 | −247.7 |
| | | | 200 | 38.7 | 3.6 | 2.1 | 3 | 34.8 | −347.1 |
| | | | | | | | | 41.8 | −36.5 |
| | | | | | | | | 39.4 | −707.7 |
| | | | 500 | 100.0 | 0.0 | 0.0 | 3 | 100.0 | −996.8 |
| | | | | | | | | 100.0 | −87.2 |
| | | | | | | | | 100.0 | −1796.9 |

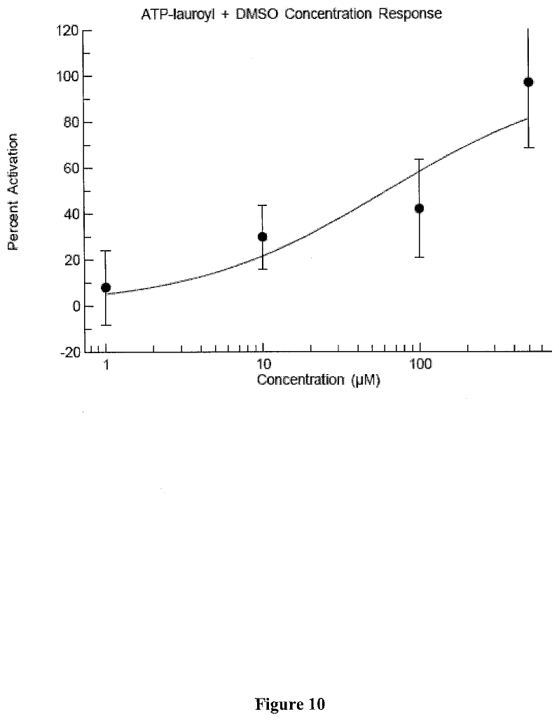

A comparison of the P2X7 receptor kinetic data between ATP and BzBz-ATP shows a slight improvement of receptor activation efficacy using the BzBz-ATP derivative, confirming previous reports.
Various embodiments of the present invention comprising ATPds were then tested. For example, a DMSO solubilized lauroyl-ATP (lauroyl-ATP is observed to be only partially soluble in aqueous solution). See, Table 3 and FIG. 10.

TABLE 3
P2X7 Receptor Activation Characteristics Using lauroyl-ATP (DMSO).

| | | | | | | | | Individual | |
|---|---|---|---|---|---|---|---|---|---|
| Test | | | | Mean % | | | | Data | Current |
| Article | | EC₅₀ | Conc | P2X7 | Standard | Standard | | Points (% | Amplitude |
| ID | Lot | (μM) | (μM) | Activation | Deviation | Error | n | Activation) | (pA) |
| ATP- | JH-0F4-07 | 62.356 | 1 | 8.1 | 16.1 | 9.3 | 3 | 26.6 | −213.4 |
| lauroyl | | | | | | | | −0.3 | 2.4 |
| | | | | | | | | −2.1 | 23.0 |
| | | | 10 | 29.9 | 13.8 | 8.0 | 3 | 45.1 | −361.9 |
| | | | | | | | | 18.1 | −146.9 |
| | | | | | | | | 26.4 | −286.0 |
| | | | 100 | 42.0 | 21.3 | 12.3 | 3 | 38.7 | −309.9 |
| | | | | | | | | 64.8 | −525.1 |
| | | | | | | | | 22.6 | −244.4 |
| | | | 500 | 96.9 | 28.5 | 16.5 | 3 | 98.3 | −788.4 |
| | | | | | | | | 124.6 | −1010.2 |
| | | | | | | | | 67.7 | −733.7 |

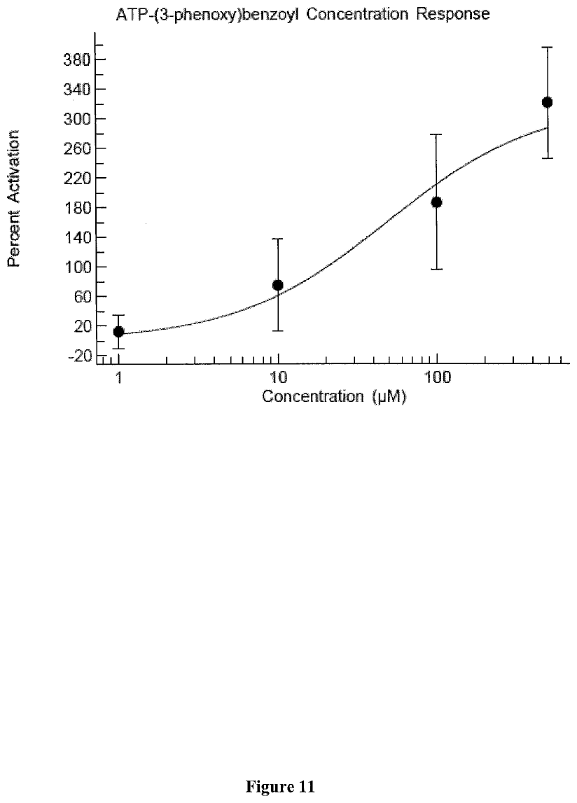

It can be seen that a dramatic decrease in EC₅₀ is observed with this particular ATPd when compared to either native ATP (˜580%) or BzBz-ATP (˜350%). Consequently, a fully soluble lauroyl-ATP derivative demonstrates vastly superior P2X7 receptor activation characteristics in comparison to these two previously reported compounds. Similarly, another ATPd, 3-phenoxybenzoyl-ATP also demonstrates improved P2X7 receptor activation characteristics. See, Table 4 and FIG. 11.

TABLE 4
P2X7 Receptor Activation Characteristics Using 3-phenoxybenzoyl-ATP.

| | | | | | | | | Individual | |
|---|---|---|---|---|---|---|---|---|---|
| Test | | | | Mean % | | | | Data | Current |
| Article | | EC₅₀ | Conc | P2X7 | Standard | Standard | | Points (% | Amplitude |
| ID | Lot | (μM) | (μM) | Activation | Deviation | Error | n | Activation) | (pA) |
| ATP-(3- | JH-OF4-05 | 49.059 | 1.00 | 12.3 | 23.4 | 13.5 | 4 | 6.7 | −3.7 |
| phenoxy)benzoyl | | | | | | | | −9.7 | 2.8 |
| | | | | | | | | 7.0 | −2.9 |
| | | | | | | | | 45.4 | −58.8 |
| | | | 10.0 | 74.7 | 62.4 | 36.0 | 4 | 36.6 | −20.3 |
| | | | | | | | | 167.5 | −47.7 |
| | | | | | | | | 39.7 | −16.3 |
| | | | | | | | | 54.9 | −71.0 |
| | | | 100 | 186.3 | 91.1 | 52.6 | 4 | 85.6 | −47.5 |
| | | | | | | | | 284.3 | −80.9 |
| | | | | | | | | 136.9 | −56.3 |
| | | | | | | | | 238.4 | −308.5 |
| | | | 500 | 320.8 | 74.8 | 43.2 | 4 | 346.4 | −192.3 |
| | | | | | | | | 357.0 | −101.6 |
| | | | | | | | | 209.5 | −86.1 |
| | | | | | | | | 370.2 | −479.0 |

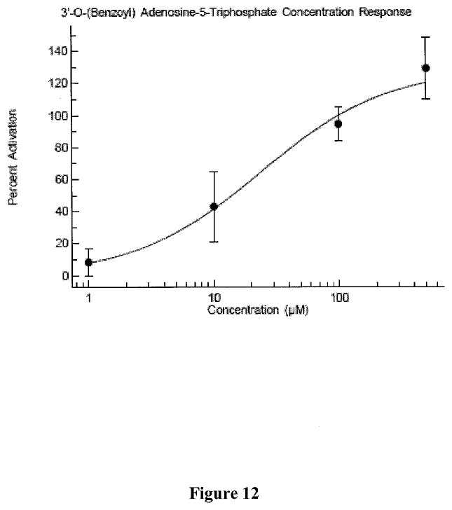

It can be seen that a dramatic decrease in EC₅₀ is observed with 3-phenoxybenzoyl-ATP when compared to either native ATP (˜738%) or BzBz-ATP (˜445%). Consequently, a 3-phenoxybenzoyl-ATP derivative demonstrates vastly superior P2X7 receptor activation characteristics in comparison to these two previously reported compounds. Similarly, another ATPd, benzoyl-ATP also demonstrates improved P2X7 receptor activation characteristics. See, Table 5 and FIG. 12.

TABLE 5
P2X7 Receptor Activation Characteristics Using benzoyl-ATP.

| | | | | | | | | Individual | |
|---|---|---|---|---|---|---|---|---|---|
| Test | | | | Mean % | | | | Data | Current |
| Article | | EC₅₀ | Conc | P2X7 | Standard | Standard | | Points (% | Amplitude |
| ID | Lot | (μM) | (μM) | Activation | Deviation | Error | n | Activation) | (pA) |
| 3′-O- | JH-0F4-04 | 24.007 | 1 | 8.3 | 8.3 | 4.8 | 3 | 9.7 | −9.9 |
| (Benzoyl) | | | | | | | | −0.7 | 1.4 |
| Adenosine-5- | | | | | | | | 15.8 | −5.4 |
| Triphosphate | | | 10 | 43.0 | 22.1 | 12.7 | 3 | 65.4 | −66.4 |
| | | | | | | | | 42.3 | −88.7 |
| | | | | | | | | 21.3 | −7.3 |
| | | | 100 | 94.5 | 10.8 | 6.2 | 3 | 102.5 | −104.0 |
| | | | | | | | | 82.3 | −172.8 |
| | | | | | | | | 98.7 | −33.9 |
| | | | 500 | 129.2 | 19.1 | 11.0 | 3 | 148.4 | −150.5 |
| | | | | | | | | 129.1 | −271.0 |
| | | | | | | | | 110.2 | −37.9 |
| BzBzATP | 050M5050V | | 200 | 100.0 | 0.0 | 0.0 | 3 | 100.0 | −101.4 |
| | | | | | | | | 100.0 | −209.9 |
| | | | | | | | | 100.0 | −34.4 |

It can be seen that a dramatic decrease in EC₅₀ is observed with benzoyl-ATP when compared to either native ATP (˜1511%) or BzBz-ATP (˜910%). Consequently, a benzoyl-ATP derivative demonstrates vastly superior P2X7 receptor activation characteristics in comparison to these two previously reported compounds.

The above values obtained with BzBz-ATP are in line with known values previously reported in the literature. Although it is not necessary to understand the mechanism of an invention, it is believed that ATPds induce intracellular changes in electropotential indicative of P2X7 activation. For example, the above data show that all three 3-O-ribose monoester derivatives have lower EC₅₀ values than the endogenous ligand, ATP, and/or the previously known ligand, BzBz-ATP.

It should be noted that even when lauroyl-ATP was tested in a solution with 0.3% DMSO the entire sample was still not totally dissolved. Consequently, the measured EC₅₀ value of approximately 62 μM may be underestimated. It would be expected that a solution containing a completely dissolved lauroyl-ATP derivative would show even greater P2X7 receptor activation characteristics.

### VI. Administration Of P2X₇ Receptor Agonists

P2X₇ receptor agonists can be administered to a subject by any means suitable for delivering these compounds to a subject. For example, P2X₇ receptor agonists can be administered by methods suitable enteral or parenteral administration route. Suitable enteral administration routes for the present methods include, but are not limited to, oral, rectal, or intranasal delivery. Suitable parenteral administration routes include, but are not limited to, intravascular administration (i.e., for example, intravenous bolus injection, intravenous infusion, intra-arterial bolus injection, intra-arterial infusion and catheter installation into the vasculature); peri- and intra-tissue injection (i.e., for example, peri-tumoral and intra-tumoral injection, intra-retinal injection, or subretinal injection); subcutaneous injection or deposition, including, but not limited to, subcutaneous infusion (i.e., for example, by osmotic pumps); direct application to the tissue of interest, for example by a catheter or other placement device (i.e., for example, a retinal pellet or a suppository or an implant comprising a porous, non-porous, or gelatinous material); and inhalation. Another administration route includes, but is not limited to, injection and/or infusion directly into a tumor.

Liposomes are used to deliver P2X₇ receptor agonists to a subject. Liposomes can also increase the blood half-life of the gene products or nucleic acids. Liposomes suitable for use in the invention can be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods can be used for preparing liposomes. Szoka et al., Ann. Rev. Biophys. Bioeng. 9:467 (1980); and U.S. Pat. Nos. 4,235,871, 4,501,728, 4,837,028, and 5,019,369, the entire disclosures of which are herein incorporated by reference.

The liposomes for use in the present methods can comprise a ligand molecule that targets the liposome to cancer cells. Ligands which bind to receptors prevalent in cancer cells, such as monoclonal antibodies that bind to tumor cell antigens, are preferred.

The liposomes for use in the present methods can also be modified so as to avoid clearance by the mononuclear macrophage system (“MMS”) and reticuloendothelial system (“RES”). Such modified liposomes have opsonization-inhibition moieties on the surface or incorporated into the liposome structure. In one embodiment, a liposome of the invention can comprise both opsonization-inhibition moieties and a ligand.

Opsonization-inhibiting moieties for use in preparing the liposomes of the invention are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer that significantly decreases the uptake of the liposomes by the MMS and RES. U.S. Pat. No. 4,920,016, the entire disclosure of which is herein incorporated by reference.

Opsonization inhibiting moieties suitable for modifying liposomes are preferably water-soluble polymers with a number-average molecular weight from about 500 to about 40,000 daltons, and more preferably from about 2,000 to about 20,000 daltons. Such polymers include, but are not limited to, polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups. Preferably, the opsonization-inhibiting moiety is a PEG, PPG, or derivatives thereof. Liposomes modified with PEG or PEG-derivatives are sometimes called “PEGylated liposomes.”

The opsonization inhibiting moiety can be bound to a liposome membrane. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH₃ and a solvent mixture, such as tetrahydrofuran and water in a 30:12 ratio at 60° C.

Liposomes modified with opsonization-inhibition moieties remain in the circulation much longer than unmodified liposomes. For this reason, such liposomes are sometimes called “stealth” liposomes. Stealth liposomes are known to accumulate in tissues fed by porous or “leaky” microvasculature. Thus, tissue characterized by such microvasculature defects, for example solid tumors, will efficiently accumulate these liposomes. Gabizon, et al., Proc. Natl. Acad. Sci., USA, 18:6949-6953 (1988). In addition, the reduced uptake by the RES lowers the toxicity of stealth liposomes by preventing significant accumulation of the liposomes in the liver and spleen. Thus, liposomes that are modified with opsonization-inhibition moieties are particularly suited to deliver the miRNA gene products or miRNA gene expression inhibition compounds (or nucleic acids comprising sequences encoding them) to tumor cells.

### VI. P2X₇ Receptor Agonist Pharmaceutical Formulations

P2X₇ receptor agonists compounds are preferably formulated as pharmaceutical compositions, sometimes called “medicaments,” prior to administering to a subject. Pharmaceutical compositions of the present invention are characterized as being at least sterile and pyrogen-free. As used herein, “pharmaceutical formulations” include, but are not limited to, formulations for human and veterinary use. Methods for preparing pharmaceutical compositions of the invention are described. In: Remington's Pharmaceutical Science, 17th ed., Mack Publishing Company, Easton, Pa. (1985), the entire disclosure of which is herein incorporated by reference.

Pharmaceutical formulations contemplated by the present invention comprise at least one P2X₇ receptor agonist (e.g., 0.1 to 90% by weight), or a physiologically acceptable salt thereof, mixed with a pharmaceutically-acceptable carrier. Pharmaceutical formulations of the invention may also comprise at least one P2X₇ receptor agonist which may be encapsulated by liposomes and/or a pharmaceutically-acceptable carrier. In one embodiment, a pharmaceutical composition comprises an P2X₇ receptor agonists including, but not limited to, BzBzATP. Preferred pharmaceutically-acceptable carriers are water, buffered water, normal saline, 0.4% saline, 0.3% glycine, hyaluronic acid and the like.

Pharmaceutical compositions of the invention can also comprise conventional pharmaceutical excipients and/or additives. Suitable pharmaceutical excipients include, but are not limited to, stabilizers, antioxidants, osmolality adjusting agents, buffers, and pH adjusting agents. Suitable additives include, but are not limited to, physiologically biocompatible buffers (e.g., tromethamine hydrochloride), additions of chelants (such as, for example, DTPA or DTPA-bisamide) or calcium chelate complexes (such as, for example, calcium DTPA, CaNaDTPA-bisamide), or, optionally, additions of calcium or sodium salts (for example, calcium chloride, calcium ascorbate, calcium gluconate or calcium lactate). Pharmaceutical compositions of the invention can be packaged for use in liquid form, or can be lyophilized.

For solid pharmaceutical compositions of the invention, conventional nontoxic solid pharmaceutically-acceptable carriers can be used including, but not limited to, pharmaceutical grades of mannitol, lactose, starch, magnesium stearate, sodium saccharin, talcum, cellulose, glucose, sucrose, magnesium carbonate, and the like.

For example, a solid pharmaceutical composition for oral administration can comprise any of the carriers and excipients listed above and 10-95%, preferably 25%-75%, of the at least one P2X₇ receptor agonist. A pharmaceutical composition for aerosol (inhalational) administration can comprise 0.01-20% by weight, preferably 1%-10% by weight, of the at least one P2X₇ receptor agonist encapsulated in a liposome as described above, and a propellant. A carrier can also be included as desired; e.g., lecithin for intranasal delivery.

### EXPERIMENTAL

### Example I

### Preparation of Non-BenzoylBenzoyl-Adenosine Triphosphate Derivatives

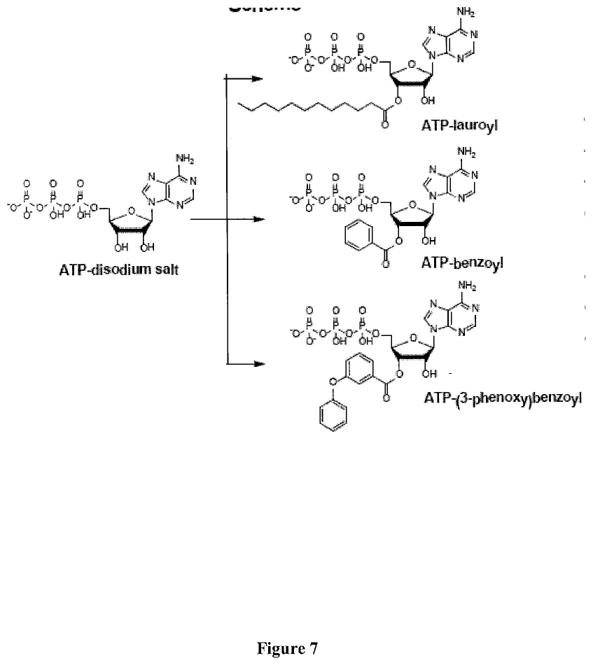

The ATPds were prepared using the starting material of an adenosine triphosphate disodium salt. Commonly known synthesis procedures resulted in the synthesis and isolation of lauroyl-ATP, benzoyl-ATP and 3-phenoxybenzoyl-ATP. See, FIG. 7. The preliminary synthesis methods resulted in a yield of approximately 100 mg of each ATPd with a purity of >95%.

### Example II

### Patch Clamp Assay Determination of ATPd Efficacy

A. Cultured Test Cells

Cells were maintained in tissue culture incubators. Cell stocks are maintained in cryogenic storage. Cells used for electrophysiology will be plated in plastic culture dishes. The tested cells were Homo sapiens HEK293 cells derived from the kidney and transformed with adenovirus 5 DNA.

B. Culture Procedures

HEK293 cells were stably transfected with the appropriate ion channel cDNA encoding the pore-forming channel subunit. Stable transfectants were selected using the G418-resistance gene incorporated into the expression plasmid. Selection pressure was maintained with G418 in the culture medium. Cells were cultured in Dulbecco's Modified Eagle Medium/Nutrient Mixture F-12 (D-MEM/F-12) supplemented with 10% fetal bovine serum, 100 U/mL penicillin G sodium, 100 μg/mL streptomycin sulfate and 500 μg/mL G418. P2X7 cells were thawed at Passage #25. The thawed cell lines are typically cultured for 30 passages.

C. Test Methods

All experiments were performed at room temperature. Each cell acted as its own control. Three concentrations of each ATPd were applied to naïve cells (n≥3, where n=the number cells/concentration). Duration of exposure to each ATPd concentration was five (5) seconds.

A positive control of 200 μM BzBzATP was applied to each cell after the application of ATPd to confirm sensitivity of the P2X7 channel and establish a reference response to which the ATPd response was compared. A vehicle control (137 mM NaCl; 4 mM KCl; 1.8 mM CaCl2; 10 mM HEPES; and 10 mM Glucose) was also be performed using vehicle to reestablish the unactivated state of the P2X7 channel prior to testing of the next ATPd.

In preparation for a recording session, an intracellular solution (130 mM K-Asp; 5 mM MgCl2; 5 mM EGTA and 10 mM HEPES) into the intracellular compartments with a planar electrode. Cell suspension was pipetted into the extracellular compartments with a planar electrode. After establishment of a whole-cell configuration, membrane currents were recorded using dual-channel patch clamp amplifiers in a PatchXpress® or Qpatch HT® system. Before digitization, the current records will be low-pass filtered at one-fifth of the sampling frequency.

Valid whole-cell recordings met the following criteria:

1. Membrane resistance (Rm) ≥200 MΩ.

Leak current ≤95% channel current.

Cells expressing P2X7 receptors were tested with an application of vehicle for 5 seconds whereupon the external solution wash out was for 25 seconds. This was done three times. ATPds were then be applied for 5 seconds and washed out in a manner similar to the vehicle. Each concentration was applied three times during the course of the run for a total of 12 ATPd applications each run. Finally, three applications of BzBzATP (200 μM) was used to evoke P2X7 current and serve as the positive control. The cells were maintained at a holding voltage of −80 mV. ATPd activation was reported as a percentage of BzATP activation (or in absolute current values).

Data was stored on a computer network for offline analysis. Data acquisition and analyses was performed using conventional software. EC₅₀ curves were prepared with Y-axis describing current change (ΔnA) application of the ATPd as a function of concentration.

1. A method, comprising:
a) providing:
i) a subject comprising at least one cancer cell; and
ii) a composition comprising a non-benzoylbenzoyl adenosine triphosphate derivative (ATPd); and
b) administering said composition to said subject wherein said at least one cancer cell undergoes apoptosis.

2. The method of claim 1, wherein said at least one cancer cell is a malignant cancer cell.

3. The method of claim 1, wherein said at least one cancer cell is a benign cancer cell.

4. The method of claim 1, wherein said at least one cancer cell comprises a papilloma cancer cell.

5. The method of claim 1, wherein said at least one cancer cell comprises an epithelial cancer cell.

6. The method of claim 1, wherein said ATPd is a 3-O-ribose monoester ATPd.

7. The method of claim 1, wherein said ATPd is selected from the group consisting of benzoyl-ATP, lauroyl-ATP, phenoxybenzoyl-ATP, and cinnamoyl-ATP.

8. The method of claim 1, wherein said administering comprises a local administration selected from the group consisting of topical, intradermal, intratumoral, intranasal and transdermal.

9. The method of claim 1, wherein said administering comprises a parenteral administration selected from the group consisting of intraperitoneal, intravenous, intramuscular and subcutaneous.

10. The method of claim 1, wherein said administering is oral.

11. A method, comprising:
a) providing:
i) a subject comprising at least one tumor; and
ii) a composition comprising a non-benzoylbenzoyl adenosine triphosphate derivative (ATPd); and
b) administering said composition to said subject wherein said at least one tumor undergoes a regression.

12. The method of claim 11, wherein said regression is partial.

13. The method of claim 12, wherein said partial regression is between approximately 10% to 90%.

14. The method of claim 11, wherein said regression is complete.

15. The method of claim 14, wherein said complete regression is 100%.

16. The method of claim 11, wherein said at least tumor is a malignant tumor.

17. The method of claim 11, wherein said at least tumor is a benign tumor.

18. The method of claim 11, wherein said at least one tumor comprises a papilloma.

19. The method of claim 11, wherein said at least tumor comprises an epithelial tumor.

20. The method of claim 11, wherein said ATPd is a 3-O-ribose monoester ATPd.

21. The method of claim 11, wherein said ATPd is selected from the group consisting of benzoyl-ATP, lauroyl-ATP, phenoxybenzoyl-ATP, and cinnamoyl-ATP.

22. The method of claim 11, wherein said administering comprises a local administration selected from the group consisting of topical, intradermal, intratumoral, intranasal and transdermal.

23. The method of claim 11, wherein said administering comprises a parenteral administration selected from the group consisting of intraperitoneal, intravenous, intramuscular, subcutaneous.

24. The method of claim 11, wherein said administering is oral.

25-42. (canceled)
